(19) United States
(12) Patent Application Publication
(10) Pub. No.: US 2023/0274809 A1
(43) Pub. Date: Aug. 31, 2023

(54) SYSTEMS AND METHODS FOR MANAGING CLINICAL PATHWAYS AND TREATMENT PLANS

(71) Applicant: KONINKLIJKE PHILIPS N.V., EINDHOVEN (NL)

(72) Inventors: Nevenka DIMITROVA, PELHAM MANOR, NY (US); Shai KREMER, HAIFA (IL); Nadav SHARABI, PETAH TIQWA (IL); Ronen SOLOMON, PETAH TIQWA (IL); Olena MARCHENKO, VALHALLA, NY (US); Moran BENTZUR, PETAH TIQWA (IL); Irit BEN-AVRAHAM, PETAH TIQWA (IL); Nasser RAWASHDEH, HAIFA (IL); Elia ROHANA, HAIFA (IL)

(21) Appl. No.: 18/012,268

(22) PCT Filed: Jun. 21, 2021
(86) PCT No.: PCT/EP2021/066846

§ 371 Date: Dec. 22, 2022

Related U.S. Application Data
(60) Provisional application No. 63/046,867, filed on Jul. 1, 2020.

(51) Int. Cl. G16H 20/00; G16H 10/60; G06F 3/0482; G06F 30/12; G16H 15/00

(52) U.S. Cl. CPC G16H 20/00; G16H 10/60; G06F 3/0482; G06F 30/12; G16H 15/00

(57) ABSTRACT

Cancer care is increasingly more complicated due to increasing therapeutic options and is often difficult to access information critical to clinical decisions as a result of the frequency of new studies and decentralization of complex clinical evidence. Patient genomic and other clinical information is critical for making timely and accurate oncology and health care decisions is often in narrative text format scattered across multiple reports and IT systems. It is critical for treatment to encode clinical knowledge pertinent to clinical decision making in an easily accessible form. Discretized information in a digital patient file can assist in creating and selecting relevant therapies based on the body of medical and genomic testing history for a given patient. Discretized information is linked to a pathway execution engine and dynamically updated clinical trial matches. An authoring tool assists in creating pathways for the pathways execution engine to drive care through clinical decision trees.

### FIELD

Various embodiments disclosed herein relate to clinical enterprise and practice management systems and, more specifically, but not exclusively, to systems and methods for managing patient treatment by clinicians.

### BACKGROUND

In recent years, there has been tremendous growth in oncology clinical pathways by providers and payers. It is widely recognized that the adoption of clinical pathways is helping to standardize cancer care process and enable adherence to established clinical protocols in cancer centers and affiliated hospitals and private practices. Employing clinical pathways impacts efficacy, safety, and overall cost of care. Many large insurance companies also partner with oncology providers and pathway companies to implement oncology pathways as a means for both reducing variation and helping in controlling costs associated with cancer diagnosis and treatment. Moreover, clinical pathways and similar tools are proving similarly useful in other fields of care such as cardiology, radiology, and pathology.

### SUMMARY

In one example, a computer implemented method for generating a clinical pathway includes creating a new data object for representing the clinical pathway, displaying a graphical tool to a user, receiving, via the graphical tool, an identification of a first node to add to the clinical pathway, updating the graphical tool to include a graphical representation of the first node, receiving, via the graphical tool, an identification of a patient status in association with the first node, updating the data object to include data representing the first node and the identification of the patient status, and storing the data object in a database in association with an identification of a disease for future retrieval.

In one example, the computer implemented method for generating a clinical pathway further includes receiving, via the graphical tool, an identification of a second node to add to the clinical pathway, receiving, via the graphical tool, a first characteristic in association with the second node, wherein the first characteristic defines criteria for determining applicability of the second node to a patient, and updating the data object to include data representing the second node and the first characteristic.

In one example, the computer implemented method for generating a clinical pathway further includes displaying, via the graphical tool, a menu for selecting criteria including a plurality of selectable criteria options, wherein the step of receiving the first characteristics comprises receiving a selected criteria option from the plurality of selectable criteria options selected by the user on the menu for selecting criteria.

In one example, the computer implemented method for generating a clinical pathway further includes displaying the menu for selecting criteria including retrieving a plurality of ontological values from an ontology, and presenting the plurality of ontological values as at least a portion of the plurality of selectable criteria options.

In one example, the computer implemented method for generating a clinical pathway further includes receiving, via the graphical tool, an identification of an additional node to add to the clinical pathway, receiving, via the graphical tool, an identification of a treatment in association with the additional node, and updating the data object to include data representing the additional node and the identification of a treatment.

In one example, the computer implemented method for generating a clinical pathway further includes the identification of a treatment including an identification of a clinical trial.

In one example, the computer implemented method for generating a clinical pathway further includes submitting the data object to a review process, wherein the review process includes one or more reviewing authorities one of approving the data object for publication or suggesting revisions to the data object, wherein the data object is not published until the one or more reviewing authorities have approved.

In one example, the computer implemented method for generating a clinical pathway further includes receiving a discipline identification, and updating the data object to include the discipline identification.

In one example, the computer implemented method for generating a clinical pathway further includes the discipline identification including one of medical oncology or radiation oncology.

In one example, the computer implemented method further includes the graphical tool including a computer assisted design (CAD) interface provided through a web front end.

In one example, the computer implemented method for generating a clinical pathway further includes accessing a worklist of pathways, regimen, and treatment plans associated with a user, wherein the data object is saved to the worklist and the user accesses the data object through the worklist to generate additional updates.

In one example, a computer-implemented method for treating a patient with a clinical pathway includes accessing patient information including one or more of diagnostic information, medical history, or current status information of a patient, determining a clinical pathway for treating the patient based on the accessed patient information, generating a graph based on the determined clinical pathway, the graph including an initial node including a disease identifier, one or more decision nodes including one or more of patient characteristics, treatment conditions or pathway timing elements, and one or more terminating nodes including treatment plan information or a reference to a second graph associated with a second clinical pathway, navigating the graph to a terminating node based on the accessed patient information, and providing one or more treatment plan recommendations based on the navigated to terminating node.

In one example, the computer implemented method for treating a patient with a clinical pathway further includes identifying a field within a decision node that cannot be determined based on the accessed patient information, pausing navigation of the graph, prompting a user for the identified field, and resuming navigation of the graph in response to receiving the prompted for identified field.

In one example, the computer implemented method for treating a patient with a clinical pathway further includes the identified field including a genomics test result for the patient.

In one example, the computer implemented method for treating a patient with a clinical pathway further includes receiving a treatment plan selection from among the generated treatment plan recommendations, and generating one or more of one or more consent forms or a report based on the treatment plan selection.

In one example, the computer implemented method for treating a patient with a clinical pathway further includes receiving edits to the one or more of one or more consent forms or the report based on the treatment plan selection, the edits including one or more of modifications to a signature field, a signature date field, a provider field, or a signature time field.

In one example, the computer implemented method for treating a patient with a clinical pathway further includes receiving an off-pathway treatment plan specification, the off-pathway treatment plan specification including a rationale for treating the patient off-pathway.

In one example, the computer implemented method for treating a patient with a clinical pathway further includes the one or more treatment plan recommendations including one or more of a Federal Drug Administration (FDA) approved therapy, an investigational therapy, or a clinical trial.

### BRIEF DESCRIPTION OF THE DRAWINGS

In order to describe the manner in which the advantages and features of the disclosure can be obtained, a more particular description of the principles briefly described above will be rendered by reference to specific embodiments thereof which are illustrated in the appended drawings. Understanding that these drawings depict only exemplary embodiments of the disclosure, and are not therefore to be considered to be limiting of its scope, the principles herein are described and explained with additional specificity and detail through the use of the accompanying drawings in which:

FIG. 1 is a systems architecture diagram of an example of a clinical support system, in accordance with some embodiments of the subject technology;

FIG. 2 is an example of a worklist interface, in accordance with some embodiments of the subject technology;

FIG. 3 is an example of a pathway creation interface, in accordance with some embodiments of the subject technology;

FIG. 4 is an example of pathway design tool interface, in accordance with some embodiments of the subject technology;

FIG. 5A and FIG. 5B are examples of a node configuration interface, in accordance with some embodiments of the subject technology;

FIG. 6 is an example of a pathway review interface, in accordance with some embodiments of the subject technology;

FIG. 7 is a flowchart depicting an example method for authoring a clinical pathway, in accordance with some embodiments of the subject technology;

FIG. 8 is an example of a patient data view interface, in accordance with some embodiments of the subject technology;

FIG. 9 and FIG. 10 are an example of a pathways navigation interface, in accordance with some embodiments of the subject technology;

FIG. 11 is an example of a treatment selection interface, in accordance with some embodiments of the subject technology;

FIG. 12 is an example of a pathway navigation interface following selection of a plan from treatment selection interface of FIG. 11, in accordance with some embodiments of the subject technology;

FIG. 13A is an example of a therapy and clinical trials selection interface, in accordance with some embodiments of the subject technology;

FIG. 13B is an example of a treatment plan view, in accordance with some embodiments of the subject technology;

FIG. 13C is an example of a clinical trial view, in accordance with some embodiments of the subject technology;

FIG. 13D is an example of a regimen view, in accordance with some embodiments of the subject technology;

FIG. 14 is an example of consent forms and report interface, in accordance with some embodiments of the subject technology;

FIG. 15 is an example of an automatic therapy matching interface, in accordance with some embodiments of the subject technology;

FIG. 16 is a flowchart of an example method for interacting with a pathway through a clinical support system, in accordance with some embodiments of the subject technology;

FIG. 17 is a sequence diagram of an example of a business process modeling and notation engine workflow, in accordance with some embodiments of the subject technology;

FIG. 18 is a block diagram of clinical support system, in accordance with some embodiments of the subject technology;

FIG. 19 illustrates a relations and hierarchy model for pathways objects, in accordance with some embodiments of the subject technology; and

FIG. 20 is a block diagram of an example of a computing system for performing the subject technology disclosed herein.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

Generally, organizations involved in diagnosis and treatment have respective processes to design, implement, use, and perpetually revise respective clinical pathways and the role pathways play in care delivery, reimbursement, and an overall institutional mission. For example, organizations such as Dana Farber Cancer Institute, Moffitt cancer center, and various others have respective pathways disease programs defining design, implementation, and management of pathways. A pathways disease program may be run by program managers who coordinate the work of multiple disease committees. The program managers develop lists of diseases upon which to focus and respective strategies for tackling content development of respective pathways associated with the diseases as well as coordinating timing relative to all other ongoing initiatives.

Informatics tools for driving clinical pathways with context-sensitive patient medical data is a particularly challenging field in medical informatics and is often a roadblock for conducting clinical care while improving patient outcomes and staff experience and containing healthcare costs. Decision support tools have been unable to facilitate bringing relevant up-to-date clinical evidence into the hands of clinicians while they have extracted and/or discretized patient data for interpretation and creation of clinical treatment plans.

While clinical know-how may exist in the form of published clinical evidence within journals and guidelines, or as in-house clinical pathways published internally in PDF, this format is often problematic for clinical providers. It is often difficult and time-consuming to capture the reasoning of, and record adherence to, clinical pathways (guidelines). In effect, what is often absent is a clinical support system that allows for interactive creation, and modification, updating, and versioning of clinical pathways, provides for interactive access and guidance in the form of selectable treatment options and/or finding alternative options, allows for collaboration among different institutions to capture multi- and/or cross-institution best practices, which may be modified or localized for particular patient populations, and/or provides detailed adherence analysis of pathways utilization at an institutional level.

Moreover, the clinical support system may provide in-platform pathway authoring and publication utilities. Authors within the pathway disease committee may follow a process for developing clinical pathways. For example, a consensus for a specific treatment pathway may be discussed and sought among a pathways team, physicians, pharmacists, and other departments on specific steps and an overall decision tree of the specific treatment pathway. Senior members from across relevant disciplines and departments may then be invited to review the pathway and offer approval (or disapproval and revisions or comments). Once approval is obtained from the committee and record, the pathways may be documented and published.

Once the clinical pathway is decided upon, various informatics technologies may make use of the clinical pathways. To facilitate integration into informatics technologies, clinical pathways can be described by decision trees in a drawing tool (e.g., Microsoft Visio), a computer assisted design (CAD) tool, or the like, and then published using, for example and without imputing limitation, portable format such as Portable Document Format (PDF), etc. However, many such portable formats, such as PDF, are not interactive and may be very cumbersome to navigate through as a result. Additionally, it is often hard to capture decision thought processes and/or alternatives in case of a decision by the clinician to go off pathway. Analysis and maintaining awareness of implementation of pathways programs are subject to error and delay as a result of incongruous systems which often use manual review and updating to accomplish parity between the systems.

In one example, workflows supporting the decision-making process along the entire path of patient treatment can be generated and managed through informatics solutions or portals that make up at least a portion of the clinical support system. The portals can, at a point-of-care, support busy professionals in oncological diseases and oncological conditions domains. Clinicians may use the portals to, for example and without imputing limitation, access consensus-driven and evidence-based state of the art care information and studies, access electronic medical records, deliver personalized care adhering to best practices and utilizing content automatically, or semi-automatically, extracted from the electronic medical records, automate importation of data from a patient clinical record for enhanced personalized care decisions, and record, manage, and/or provide aggregate processing on treatment plans and decisions made within the portal.

As a result, clinical pathways and guidelines may be well defined and disseminated through the portals in order to optimize clinical care across all accessing clinics. While many institutions use National Comprehensive Cancer Network (NCCN) guidelines, the NCCN guidelines provide only broad direction and do not provide a well-defined framework with substantial specificity for recommendations for individual patient on a day-to-day basis. Further, clinicians may use in-house clinical pathways and/or a diverse set of in-house information technology (IT) systems in order to conduct patient care.

As disclosed herein, a clinical support system may provide functionality to keep abreast with medical literature (e.g., journals, reprints, studies having to do with advances in therapeutics, treatment protocol advancements, etc.) and provide access to consensus based, evidence-based, and state-of-the-art care through a peer reviewed curation system. While clinical pathways authoring, curation, and usage is discussed herein, it is understood that the clinical support system of this disclosure may provide similar support for medical literature and care guidance. The clinical support system can integrate and link to electronic medical records (EMRs) through API integrations and support pathways through various interfaces such as HL7 and fast health interoperability resources (FHIR) to allow for bidirectional communication (e.g., to retrieve and upload information across patient care workflows, therapies, and a relevant EMR). In effect, pathways can be constructed to deliver personalized care by combining best practices as determined through peer-review process and extract content and data elements from EMRs to improve automations and quasi-automated support. For example, patient specific comorbidities and sensitivities to drugs, individual ability of a patient to metabolize drugs, unique white blood cell characteristics, and the like may be accounted for to assist in making clinical decisions within a rigorous framework and responsive to the patient specific data and so enable clinicians to individualize and craft a treatment plan for a specific patient. Moreover, pathways usage, adherence, deviation, and the like can be recorded to produce actionable analytics as the volume of use cases of the clinical support system increases.

The standardization, automation, and streamlining of access to clinical pathways may result in improved and more efficient patient care. For example, in the case of early stage cancer, a respective clinical pathway may be more straightforward earlier than at later stages of the cancer. However, for certain types of cancer and late stage cancer, doctors often must navigate through numerous complexities when determining a care and treatment plan. Clinical pathways portals can reduce variability in clinical practice and improve patient outcomes by streamlining practice. Clinical pathways portals promote organized and efficient patient care as well as a utilization of evidence-based medicine. As a result, outcomes in oncology care settings can be optimized efficiently and effectively across a multitude of clinics (as compared to on a clinic-by-clinic basis). Effectively, clinical pathways portals perform complex processes of encoding the clinical expertise of cancer center institutes into formal clinical pathway programs. Moreover, once pathways are encoded as distinct decision trees, with appropriate clinical regimens and stepwise decision nodes, the encoded pathways can be published as, for example, PDF documents for alternative (e.g., off platform, etc.) methods of dissemination.

In one example, a clinical support system (e.g., for medical decisions, etc.) provides access to both an authoring environment and a clinical provider environment, via a pathways authoring portal and a pathways provider portal respectively. The pathways provider portal allows for assisted and/or automated pathway and treatment matching based on patient information (e.g., medical records, diagnostic information, etc.), as well as provides an automated driver for clinicians navigating a pathway workflow. The authoring environment allows for creating new regimens and treatment plans, clinical trials, and encoding decision trees based on clinical evidence.

Further, authorized users (e.g., a clinical provider, etc.), may be presented with a complete patient diagnostic picture along with response to treatments and information from electronic medical records (EMRs), picture archiving and communication system (PACS), laboratory information system (LIS), clinical phenotype, etc. Detailed biomarker status of the patient, in some cases based on genomics and molecular pathology results, can be presented to the authorized user. Different clinical decision support options may be presented to, and actively guide, a physician (or other authorized user) through decision trees encoding the current best practices and expertise of clinical pathways.

The physician interacts with the clinical pathways and treatment plans through graphical user interfaces (GUIs) providing screens for respectively viewing patient information, managing pathways, and/or navigating a selected pathway. Clinical trials may be selected as a pathway is navigated and automatically incorporated into a treatment plan, in whole or in part, or presented to the clinician as recommendations for adoption. Treatment plans may also be created electronically and supporting documents for clinical care can be signed and submitted into a respective EMR. Other data exports, of varying formats and to varying receiving services, are supported to, for example, generate analytics for assessment of the efficacy of a respective clinical pathways program and the like.

The clinical support system may combine integrated diagnostic and monitoring information processes with clinical decision support applications. Data can be gathered into a unified patient model by ingesting data from a respective EMR in a standardized and discrete format using standards such as, for example and without imputing limitation, those published by Health Level 7 (HL7) and the like. In order to ensure that sufficient information is discretized to support therapy matching and dynamic clinical trial matching, methods for discretizing clinical report data and genomics report data can be executed by an integrated service either natively to the clinical support system or through external application programming interface (API) calls. For example, many advanced stage clinical pathways benefit from detailed biomarker information which may be found in genomics and molecular pathology reports. Thus, the discretized data can be used in combination with business process modelling and notation (BPMN) engines to drive inferencing.

In one example, the clinical support system architecture includes several major components which all have many critical subcomponents or services. A workflow engine acts as a master component and interacts with a pathways authoring portal and a pathways provider portal. The pathways authoring portal provides tooling and workflow for a user to create new clinical pathways. In some examples, the pathways authoring portal provides collaborative authorship operationality to, for example, create clinical pathways by a team. The pathways provider portal provides a centralized channel for disseminating, updating, and viewing clinical pathways. In some examples, clinical pathways authored outside the tool can be imported into, and access through, the pathways provider portal.

The workflow engine may communicate with a business process modeling and notation (BPMN) engine to assist in creation and storage of decision trees directed by interactive user input through the pathways authoring portal. Once a clinical pathway is approved, the pathways provider portal may interact with the workflow engine to execute relevant pathways from a respective pathways knowledgebase in the context of individual patient clinical data (e.g., manually or automatically derived from an EMR, diagnostic devices, etc.) by invoking the BPMN engine.

In some examples, the pathways authoring portal includes a pathways authoring tool that is an end user application supporting an authoring workflow. The authoring tool is a self-service tool and enables users to create, modify, and manage various knowledge-curated items, such as, for example and without imputing limitation, regimens, treatment plans, clinical trials, and pathways decision trees.

When authoring a pathway, the user may first access a pathways worklist. The pathways worklist includes pathways currently being edited. From the pathways worklist, the user can create a new pathway or modify an existing pathway. In some examples, pathways can be built on existing treatment plans, which are in turn built on existing regimens.

New pathways can be created using a graphical tool provided through the GUI. For example, new decision nodes can be added to a new decision tree corresponding to the new pathway. Moreover, terminal nodes may be created and added to the decision tree, which link treatment plans and/or clinical trials to the decision tree or pathway. In some examples, multiple users may update the same pathway at the same institution or at collaborating institutions based on the user credentials with which they log into the pathways authoring portal. Pathways may be reviewed and/or approved by a committee before being stored in a knowledgebase. In some examples, committee review and approval may be a prerequisite for using real clinical data profiles from actual patients with a pathway.

The pathways provider portal enables a clinical provider to step through a workflow defined by a clinical pathway decision tree. The workflow engine assists in coordinating various processes, including, for example and without imputing limitation, input data ingestion, patient dashboard presentation, pathways navigation, treatment selection, and summary and sign off. The workflow engine may drive user experience (UX) actions, and input received back from the UX may drive the workflow to appropriate and sequential next stages. In some examples, each step of the workflow creates a new tab in a GUI for the UX. Additionally, input data may come from multiple information sources, such as, for example and without imputing limitation, an EMR, LIS, radiology information system (RIS), genomics system, molecular pathology system, etc.

In one example, a clinical provider can launch a clinical decision support application (e.g., a decision tree and workflow GUI) through the pathways provider portal application from a patient chart (e.g., in an EMR, etc.), a patient context display, and/or from a patient dashboard. Relevant patient data can be automatically extracted and/or manually entered during navigations and then displayed to the clinical provider through the pathways provider portal.

The provider can see current treatment plans, add a new treatment plan, or modify existing treatment plans. Further, a respective pathway(s) may be automatically navigated to and through based on entered data. For example, based on available data, the pathways portal application may display an indication of a current position of the patient's treatment within the full pathway by, e.g., highlighting or otherwise distinguishing one (or, in some cases, more than one) node of the displayed pathway. Additionally, or alternatively, the pathways portal application may identify one or more next steps to be taken in the patient's care based on the current position by, for example, distinguishing one or more nodes of the pathway or showing a list of next steps separate from the pathway. The provider may review the respective decision tree, manually complete navigation (by selecting choices or making decisions at nodes within the decision tree) through the decision tree, and/or confirm a resultant final pathways decision node. The resultant final pathways decision node may be automatically selected based on entered data and preceding navigation of the decision tree.

Matched on-pathway treatment plan options, along with clinical trials options, may be displayed to the provider according to the patient data and/or decision tree navigation selections. The provider may select from one of the matched options or an off-pathway option. A corresponding treatment plan summary report can then be generated, as well as any relevant consent forms, and provided to the provider for review and sign off. The provider can also print the forms (e.g., for patient sign off, etc.). The final treatment plan may be sent back to the EMR or an appropriate pharmacy system in a hospital for downstream preparations (e.g., preparing medications, hospital services, etc.).

In one example, the clinical support system includes an input module, a workflow engine, and a knowledgebase. The input module is configured to discretize clinical and genomics data. The workflow engine drives process forward by sequentially iterating through different states of the clinical support system. The knowledgebase includes one or more data models able to support patient data and transformable into standardized ontological representation.

The workflow engine drives various steps in clinical decision making workflow, such as, for example and without imputing limitation, retrieving patient input information, clinical pathway creation, selection of a recommendation from a terminal node within a clinical pathway, treatment plan generation, and sign off and creation of a final report document. In general, each step in the clinical decision making workflow is represented in a tabbed window and a respective GUI. The workflow engine may adapt clinical pathway creation responsive to user interaction and automatic navigation rules. Additionally, the final report document may be generated in multiple formats, including, for example and without imputing limitation, PDF, XML, JSON, and/or other structured formats. As a result, the workflow engine advances the clinical decision support system through various states, corresponding to steps in a respective workflow, and fetches new information from respective databases accordingly.

The workflow engine interacts with multiple databases to drive the workflow process with a web component to present an interactive application, or GUI, to an end user. As discussed above, the knowledgebase includes a transformable patient data model based on ontologies. The ontologies may represent, for example and without imputing limitation, diseases (e.g., ICD10, SNOMED CT02, etc.), standardized TNM staging (e.g., using American Joint Committee on Cancer (AJCC) methodology, etc.), biomarker data models utilizing standardized gene nomenclatures (e.g., Human Genome Organization (HUGO) nomenclature, etc.), standardized drug representations, and/or standardized clinical trial representations, etc.

The knowledgebase may store different types of information, including decision trees. The stored decision trees correspond to an applicable reasoning process for use in therapy planning.

In some examples, the BPMN engine can be a microservice. The BPMN engine matches patient profiles with appropriate representations in the knowledgebase. Moreover, the BPMN engine manages process workflows, such as, for example and without imputing limitation, storing authored processes, initializing process workflows, and status monitoring and alerting.

In one example, a web front end renders a clinical pathways portal where users can be authenticated and log in to perform various functions, such as, for example and without imputing limitation, pathways authoring activities (e.g., for program managers, etc.) and/or treatment planning activities for practicing clinicians (e.g., medical oncologists, surgical oncologists, radiation oncologists, etc.).

Stored authored processes may include, for example, decision trees derived from national guidelines and/or clinical pathways based upon best practices within a particular hospital. Each node within a decision tree can include condition values and action values. Actions may indicate next steps within the respective decision tree (e.g., may point to a next node, etc.), while condition values allow a user navigating the pathway to input or select relevant patient conditions. Nodes may also draw upon elements from the standardized ontologies discussed above. Additionally, initialization of process workflows may be linked to authoring of a new curated item of knowledge.

The stored decision trees can be curated by authorized users (e.g., relevant subject matter experts (SMEs), etc.). Curation functionality may include manually executable tasks, such as, for example and without imputing limitation, user assignment, updating decision trees (and other stored process workflow), and resumption of process execution, etc. Further, decision trees and other process workflow may be versioned and compatible with a process versioning system. As a result, though decision trees may evolve over time, they may be versioned and dated for posterity and tracking purposes. Additionally, decision trees and/or versions of decision trees may be associated with different institutions through a multitenant support model where each university is able to access respectively authorized decision trees on the same hosted tree.

The pathways authoring portal may be used to capture clinical pathways and/or guidelines in the knowledgebase. For example, clinicians or qualified professionals specialized in encoding clinical pathways may use the pathways authoring portal to create new decision trees or modify existing decision trees stored in the knowledgebase.

In effect, the pathways authoring portal provides for creation of curated knowledge that may then be applied to real clinical data profiles of actual patients. The authoring portal is a self-service tool, which allows users to create, modify, and manage various knowledge-curated items, such as regimens, treatment plans, clinical trials and pathways decision trees in an easy to use graphical user interface. In one example, the relations and information hierarchy between different curated items are as follows: a pathway includes one or more treatment plans and one or more clinical trials, and each individual treatment plan includes one treatment regimen.

A regimen is a description of a combination of approved drugs (e.g., approved by the U.S. Food and Drug Administration (FDA), etc.), as well as associated risks of treatment and other details. A treatment plan, such as, for example, a medical oncology treatment plan, is created based on a specific regimen. As a result, treatment plans include, and authors may add or modify via the authoring portal, detailed scheduling and administration information, such as, for example, drug dosage, route of drug administration (e.g., oral, intravenous, etc.), number of cycles, schedule for delivery, etc.

Treatment plans may include various procedures and details in accordance with the condition being treat. For example, treatment plans for surgery may include specific interventional procedures, such as robotic surgery for prostate cancer with nerve sparing. In another example, a treatment plan for radiation oncology may include radiation dosage and schedule, etc. Moreover, each treatment plan may also include published references to corresponding clinical evidence supporting usage of the treatment for the respective patient scenario.

Clinical trials describe various clinical trial details and include title, phase, principal investigator, and contact information for a respective clinical trial. In some examples, clinical trials may further include inclusion and/or exclusion criteria and hyperlinks to appropriate online sources (e.g., clinicaltrials.gov, etc.). Inclusion criteria may include descriptions of biomarkers which are among the inclusion criteria. Within the authoring portal and/or the clinical pathways portal, clinical trials can be associated with particular decision trees corresponding to pathways treatment nodes.

Pathways include decision tree as discussed above. The decision trees can be represented as BPMN trees or knowledge graphs. In particular, each pathway includes decision nodes and concludes with treatment nodes. In some examples, treatment nodes may be displayed as terminal nodes within a pathway decision tree. Further, each treatment node may contain one or more treatment plans and one or more clinical trials.

In one example, the authoring tool includes visual design tooling for constructing decision trees and defining branching logic through graphical components. The decision nodes of a pathway decision tree describe specific phenotypes or genotypes used for branching logic. As a result, a user may navigate through multiple options of a decision tree and respective decision nodes. For example, and without imputing limitation, non-small cell lung cancer may be either squamous or non-squamous, which may be represented as decision nodes. The squamous and non-squamous decision nodes may then lead to further decision nodes depending on the respective subtype of the non-small cell lung cancer. In comparison, the treatment nodes of the pathway decision tree contains detailed information on treatment options and clinical trials mapped to the respective pathway.

By using the authoring tool, users can define detailed rule-based logic for each decision node. The rules may be based on underlying data model parameters, ontologies, and value sets. Logical operators (e.g., AND, OR, NOT, etc.) may be defined between different rules. As a result, a user can, for example, define a decision node as ‘Stage II’, and/or a more complex decision node as ‘Stage I OR Stage II’.

The underlying data model parameters define interactions with an underlying data model which may employ multiple ontologies and terminology systems. For example, a cancer staging TNM and group stage values can be mapped to numerous AJCC editions and SNOMED-CT ontology may be used for cancer types values. Histological types can be defined by using, for example, an International Classification of Diseases for Oncology, Third Edition (ICD-O-3) ontology published by the World Health Organization (WHO), and this information may be mapped according to an appropriate LIS. Drugs used in the regimen may be based on, for example, a National Cancer Institute Thesaurus (NCIt) value set and/or other drug ontologies. As a result of usage of national and international open standards, data, and ontologies, data may be more effectively normalized and interoperability between the decision support system and external IT systems can be made robust.

Further, the authoring tool may include a review and approval process. In some examples, each curated item that is created is initialized to a “draft” state. Draft item may be submitted for review and approval by authorized users and then may be stored in the knowledgebase following appropriate review and approval. Curated items stored in the knowledgebase may then be made available for usage in the pathways provider portal. In some examples, the workflow engine manages the review and approval process across multiple stakeholders such as, for example and without imputing limitation, program managers (e.g., for entering regimens and treatment plans prior to starting the workflow or otherwise) and/or approval committee members to approve complete pathway information for storage in the knowledgebase and use in clinical settings.

In some examples, the knowledgebase may store multiple versions of a single pathway. Different version and/or a versioning history of a single pathway can be accessed via the authoring tool. The workflow engine may track the different pathway versions and disseminate updates to some or all of the versions accordingly.

The clinical pathways portal may include a front end application through which users may interact with pathways, treatments, etc. For example, users may navigate pathways and/or select treatments. Pathways can be selected based on the clinical pathways portal or the user determining a match between the respective pathways and a patient profile. In some examples, the frond end application provides for reviewing a patient file, automated navigation of corresponding BPMNs, treatment selection, off-pathway options review and selection, and summary report generation.

When reviewing a patient file, the reviewing user may access complete diagnostic information as well as medical history (e.g., EMRs, etc.) and current status. The user having reviewed the patient file, the workflow engine may automatically trigger the pathway navigation which, in some examples, opens a new tab in a clinical pathways portal GUI. In the new tab, the user may interact with a decision tree associated with the clinical pathway selected for the patient to enter or preview paths along the respective decision tree.

In some examples, the workflow engine initiates automated navigation through decision trees by triggering the BPMN and launching a corresponding BPMN pane which visually displays a pathway corresponding to the respective decision tree (e.g., a BPMN representation) matching with a respective patient's primary cancer type. The BPMN engine and workflow engine may then match the patient profile to structured rules (e.g., patient status requirements, treatment stage milestones, etc.) defined in the decision nodes of the displayed pathway.

The patient profile and rules definitions for the pathway are normalized to a common data model to support efficiently matching the patient profile to the structured rules. In cases where particular data elements or values are missing, the workflow engine may prompt the user to manually select an appropriate node and specify the input elements for the selected node. For example, where a genomics test value is missing, the user may be prompted to manually retrieve and enter the appropriate genomics data from a connected LIS or genomics system.

In some examples, once a user navigates to a terminal node (e.g., a treatment node) of a pathway, associated on-pathway treatment options may be automatically retrieved from the knowledgebase. The workflow engine may move workflow to a next step of the pathway and generate a new tab in the pathways provider portal GUI. The new tab displays treatment options including one or more curated treatment plans and clinical trial matches. The user may review details for each treatment plan and/or clinical trial via interacting with a corresponding icon (e.g., by mouse click, etc.) and select a treatment plan as a proposed treatment plan.

The treatment options automatically generated by the workflow engine, as discussed above, may be “on-pathway” options. In some cases, a clinician user may determine that no on-pathway options are appropriate for the patient (e.g., due to comorbidities, need for more aggressive treatment, patient preference, or other clinical reasons, etc.). In such a case, the clinician user may select an off-pathway treatment option and specify an alternative treatment plan in accordance with an appropriate rationale for deviating from the clinical pathway.

Once a treatment plan is select, additional tabs detailing clinical decision support information may be generated and provided to the clinician user. In some examples, a summary report tab may be generated and include a summary report and appropriate consent forms, based on customizable templates associated with the selected treatment plan and stored in the knowledgebase.

Communication with, and running of, the BPMN engine can be accomplished using an application programming interface (API) based framework. In effect, multiple microservices, or independent or quasi-independent compute processes, interact with each other via issuing API calls. API calls may be transmitted between services by Hypertext Transfer Protocol Secure (HTTPS) over a network (e.g., local area network (LAN), virtual local area network (VLAN), virtual private network (VPN), etc.). In particular, HTTPS requests with an appropriate URL destination may include parameters to, for example, request the BPMN engine initiate an authoring process (which may itself result in the BPMN engine making an API call over HTTPS to initialize an authoring process, etc.). In response, the BPMN engine may generate, or initiate a process to generate and provide back to the BPMN engine, relevant results such as, for example, access information to a stored pathway relevant to the original requesting service.

The BPMN engine microservice maintains interactions for driving decisions trees. In particular, the BPMN engine may include multiple computer processes as either integrated components of the BPMN microservice or as other microservices in communication with the BPMN engine (via API call and response, etc.). For example, a curation process may provide for entering new information into the knowledgebase. The knowledgebase may store models (e.g., decision trees, etc.), as well as other information and be provided as an accessible database. Front-end and web service components may provide for user interactions and GUI/UX rendering to users.

The web service component drives the end user applications (e.g., the pathway authoring portal and the pathway provider portal). The web service microservice acts as a workspace that holds relevant data and processes while defining specific work behaviors and conventions/configurations. The web service performs a web server role by answering client (end user) content requests, as discussed above, and maintaining interactions with a genomics web server and a web client.

The genomics web server receives, upon activation or by using a listener process, requests from the pathway provider portal and related applications. The web client renders decision trees in the GUI for the user to interact with and navigate. Further, the web client receives and appropriately directs commands from the user via interactions through the GUI, such as node selection and workflow navigation, etc.

As a result of the features disclosed herein, the BPMN engine and the workflow engine may interact to provide a novel workflow service that interacts with the front end services (e.g., UI, UX, GUI, etc.) and the BPMN engine to access decision tree data (e.g., entity trees, etc.) stored in the knowledgebase. From a user perspective, the front end initiates and drives the workflows. The workflow engine provides automations to run and match existing decision trees to provide clinical decision support.

One example of a workflow includes selection of a curation item, data retrieval for processing, starting matching processes, maintaining execution history, resumption of an ongoing process, and retrieving and/or updating status. Selecting a curation item may include providing a set of filters to the curation service of the BPMN engine, which then executes corresponding matching and fetches a curation item (e.g., a pathway identifier, etc.).

To retrieve data, the client computer (e.g., the user computer) compiles the data needed for executing the corresponding process. Details from the curation item can accessed, for example, from the decision tree nodes and terminals. Curation item details may include, for example and without imputing limitation, treatment plan data and the like. Additionally, patient data (e.g., FHIR, minimal common oncology data elements (mCODE), cancer tracking outcomes and analysis (COTA) nodal address (CNA), etc.) can be retrieved and/or accessed as well to perform the process of matching.

Having compiled the relevant data, matching can then be initiated from the front end by the user. A start request is transmitted to the BPMN engine including a curation item identifier and the compiled data as discussed above. The front end may then receive an execution history which can be mapped to a respective decision tree (e.g., pathway, guideline, etc.). The user may then manually enter information for executing decision nodes within the mapped decision tree. Where manual input is needed for a decision node, the workflow engine can pause processing until the necessary input is provided, at which point the BPMN engine may continue executing content related to the decision tree. Moreover, progress through the pathway can be updated manually by the user and currents status can be retrieved (e.g., without having to navigate through the decision tree).

The BPMN engine deploys and drives patient pathways in different modes. In manual mode, the BPMN engine assists a clinician or otherwise qualified person (e.g., authorized user) through an interactive session to select a clinical pathway based on manual input. In a fully automated mode, the BPMN engine selects a clinical pathway based on automatically stored detailed patient information including, for example, clinical data abstracted from various clinical reports (e.g., radiology, pathology, genomics, etc.) as discussed above. As a result, clinical care providers can automatically include relevant data that has been accumulated over time. In semi-automated mode, the BPMN engine prompts a user for input when the patient data lacks a critical information element for driving a decision to a next step. As a result, patients can be more efficiently transferred from outside clinics when a complete medical history for the patient is missing.

The disclosure now turns to a discussion of example embodiments for purposes of understanding and explanation. It is understood that the discussed examples are provided for explanatory, non-limiting purposes and a person having ordinary skill in the art will understand that variations and modifications of the examples discussed herein may be implemented without departing from the scope and spirit of the disclosure.

In a first example, a clinical decision for chronic myelogenous leukemia (CIVIL) made through the clinical pathways portal is described. The clinical decision may be made based on guidelines and clinical pathways in light of biomarker descriptions represented as forking events within a decision tree and an individual patient's case characteristics. For example, a clinical pathway for CIVIL stratifies along first line and second line therapies and ABL1 sensitizing mutations of the respective protein coding gene, namely Y253H. Based on this information in the BPMN representation of one or more pathways and the respective patient profile representation, the BPMN engine automatically matches the patient to treatment options and/or clinical trials, and accordingly presents the clinician with a suggestion through the clinical pathways portal GUI.

The GUI may provide the clinician with patient diagnostic information, medical history, and current status. Where, as in the example above, the patient is non-responsive to a first line therapy and has particular mutations of the ABL1 gene, the GUI provides this information to the clinician in one pane of a window or tab of the GUI. An adjacent pane shows selected steps corresponding to an already traversed arm of the respective pathway, which are appropriate for the current patient at hand based on the traversed arm(s) and the particular patient diagnostic information and medical history. The clinician user may then navigate the decision tree by selecting next nodes along the current decision path to explore potential future pathway options and the like.

In a second example, consider a patient with metastatic colorectal cancer and an unresectable tumor. Further, the patient is a candidate for first line therapy and has a genomic report available. The clinical support system disclosed herein may parse the genomics report and record (e.g., store in memory, etc.) that this patient has KRAS G12V mutation, which is a predictive biomarker associated with reduced responsiveness to certain therapies.

As a result, an associated decision tree leads a clinician user to options for Bevacizumab, which, in this example, is preferred by the clinician. A further decision point includes consideration of a performance score for the patient's response to treatment. For example, the patient may have an Eastern Cooperative Group (ECOG) score of “0” or “1.” This decision point may lead to an end node in the decision tree, which includes Capecitabine, Oxaliplatin, and Bevacizumab as a combination therapy for the patient.

Upon user confirmation, the navigated pathway may be applied to the knowledgebase and appropriate treatment plans are presented to the clinician user for review. The clinician may then select from the on-pathway options presented, or may instead create an off-pathway option. The on-pathway options may include multiple predefined options for the selected pathway. Once a treatment plan is selected, adjustments may be made to a corresponding dose and schedule. The user clinician then confirms (e.g., by clicking a “submit” button or the like) the updated treatment plan and the workflow engine may advance the clinical support system to a report and accompanying documents generation process. Alternatively, the clinician user may instead create an off-pathway option based on, for example, a patient need for more aggressive therapy, patient comorbidities, or patient preference.

A discussion of certain example embodiments of the subject technologies follows. While architectures, systems, methods, and/or apparatuses are disclosed, a person having ordinary skill in the art will understand these to be examples for general understanding and clarity. Accordingly, variations on the disclosed embodiments, such as more or fewer steps, alternative architectures, and other variations may yet still fall within the spirit and scope of the disclosure.

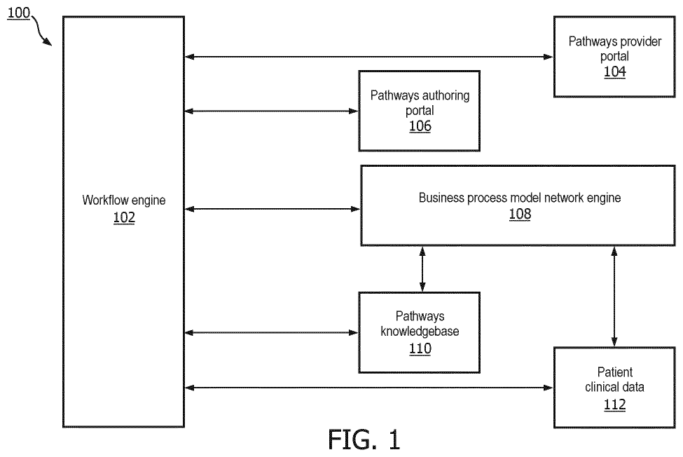

FIG. 1 depicts a systems architecture 100 for an example clinical support system. As depicted here, systems architecture 100 includes various components which may be realized by a variety of architectural approaches. For example, and without imputing limitation, systems architecture 100 may be any of a monolith, microservices network, disaggregated components mesh, or a combination of the aforementioned architectural approaches. Moreover, systems architecture 100 may be provided, wholly or partially, via servers, virtual machines (VMs), etc., on local hosts, remote hosts, cloud providers, or a combination of the aforementioned hosting options.

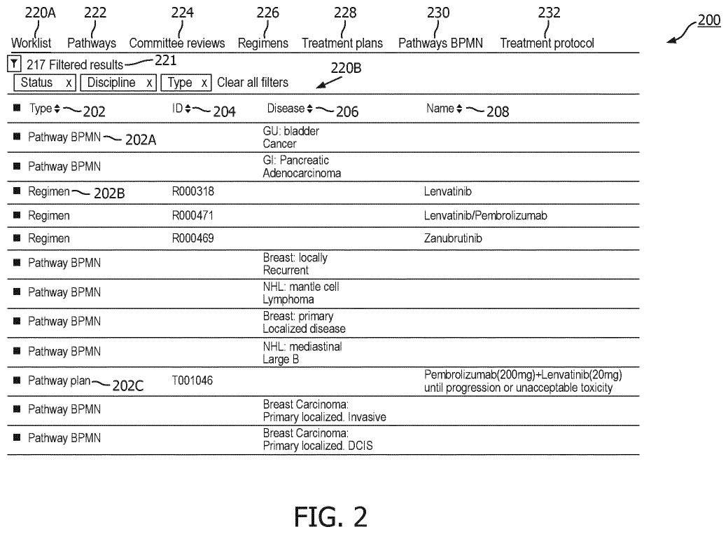

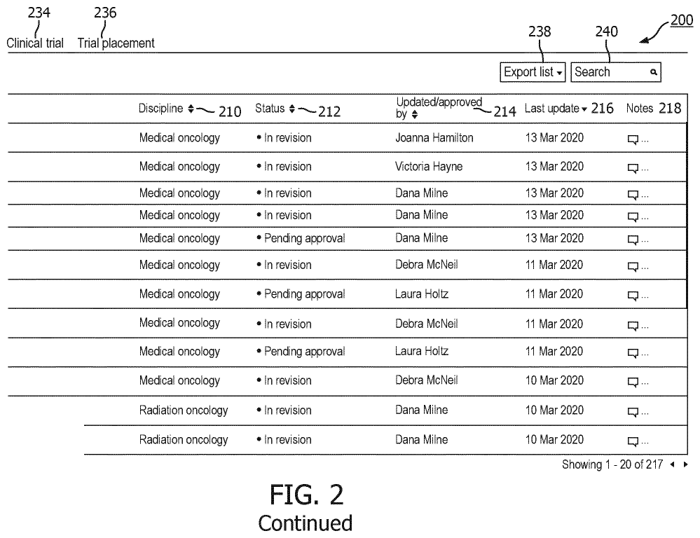

Systems architecture 100 includes a workflow engine 102, which interacts with a pathways provider portal 104, a pathways authoring portal 106, and a business process modeling and notation (BPMN) engine 108. Workflow engine 102 drives and manages processes across pathways provider portal 104, pathways authoring portal 106, and BPMN engine 108. In some examples, workflow engine 102 receives inputs from users through a graphical user interface (GUI) (discussed below in reference to FIGS. 2-6B and 8-15), and includes, or interfaces with, system controller and/or dispatcher components (not depicted).

Pathways authoring portal 106 provides an end user application for creating, modifying, reviewing for approval, and publishing pathways to be used for treating patients. FIG. 2-6B depict example GUIs for interacting with pathways authoring portal 106. In particular, FIG. 2 depicts a view 200, provided by pathways authoring portal 106, through which a user can access and author new and/or in-revision pathways. View 200 provides users an effective and intuitive listing of current items being worked on and/or awaiting approval for publishing to pathways provider portal 104.

View 200 includes a navigation bar for accessing various listings of components of pathways and/or a worklist query results 220B, which is accessed through a worklist button 220A. The listings for the other pathway components may be respectively accessed through a pathways button 222, a committee reviews button 224, a regimens button 226, a treatment plans button 228, a pathways graph button 230, a treatment protocol button 232, a clinical trial button 234, and a trial placement button 236. In effect, each respective button 220A-236 can be interacted with to navigate to a corresponding view populated with information in a pathways knowledgebase 110 (e.g., a database, data store, file system, etc.).

Moreover, respective pathway components can be created and curated through the corresponding buttons 226-234. For example, regimens button 226 allows a user to create and curate individual regimen for use in pathways, treatment plans button 228 allows a user to create and curate individual treatment plans for use in pathways, pathways graph button 230 allows a user to create and curate navigable graphs for use in pathways based treatment of a patient, treatment protocol button 232 allows a user to create and curate treatment protocols for use in pathways, and clinical trial button 234 allows a user to curate and detail clinical trials that may be used in pathways.

Here, worklist query results 220B is shown and includes a filter 221, export button 238, and search field 240. Users may filter worklist query results 220B via filter 221 (e.g., filter according to status, discipline, item type, etc.). As depicted in FIG. 200, worklist query results 220B is unfiltered and lists work items of various types, including pathway graphs 202A, regimen 202B, and treatment plans 202C. Worklist query results 220B can be exported in a local file format (e.g., .csv, .pdf, .xcl, XML, etc.) via export button 238. Moreover, users may search within worklist query results 220B by entering text in search field 240.

Worklist query results 220B is displayed in a tabular, or grid, format, in which each row is a worklist entry (e.g., a pathway 202A, regimen 202B, or treatment plan 202C). Each column denotes a particular characteristic of a respective worklist entry and, based on the worklist entry, may hold no value or a null value. That is, entries with no value are left blank or empty. Columns, and so worklist item characteristics, include type 202, identifier 204, disease 206, name 208, discipline 210, status 212, updated/approved by 214, date of last update 216, and notes 218.

Type 202 corresponds to one of pathways 202A, regimen 202B, or treatment plans 202C. Regimen 202B and treatment plans 202C may be associated with an identifier 204, and so those worklist items include an alphanumeric serial value in the corresponding field, such as “R000318,” “T001046,” etc. The worklist items associated with an identifier 204 are also associated with a name 208, which can be a common name or the like used to colloquially reference a corresponding regimen 202B or treatment plan 202C. For example, and without imputing limitation, each regimen 202B may include a respective name 208 “Lenvatinib,” “Lenvatinib/Pembrolizumab,” or “Zanubrutinib,” etc. Treatment plan 202C names 208 often include additional descriptive terms including dosage guidance and the like, such as, for example and without imputing limitation, “Pembrolizumab (200 mg)-Lenvatinib (20 mg) Until Progression or Unacceptable Toxicity).”

In comparison, pathways 202A may be associated with a particular disease 206 (whereas regimen 202B and treatment plans 202C are not directly associated with a particular disease 206), and so those worklist items include a disease name in the corresponding field. For example, and without imputing limitation, a field for disease 206 may list one of “GU: Bladder Cancer,” “GI: Pancreatic Adenocarcinoma,” “Breast: Locally Recurrent,” “NHL: Mantle Cell Lymphoma,” “Breast: Primary Localized Disease,” “NHL: Mediastinal Large B,” “Breast Carcinoma: Primary Localized Invasive,” or “Breast Carcinoma: Primary Localized DCIS.”

Discipline 210 denotes which practice domain the corresponding worklist item falls within. For example, and without imputing limitation, discipline 210 may include “Medical Oncology” or “Radiation Oncology.” Status 212 denotes where the corresponding worklist item is located within a workflow. For example, worklist items that are still being constructed and/or modified may be listed as “in revision” while worklist items that have been submitted for review and/or approval may be listed as “pending approval.” Updated/approved by 214 denotes the name of the last user to modify the corresponding worklist item, where the worklist item is “in revision,” or the name of the user responsible for reviewing and approving the worklist item, where the worklist item is “pending approval.” Date of last update 216 denotes when the corresponding worklist item was last edited or reviewed.

Notes 218 provides a field for users to enter text notes related to the corresponding worklist item, for later self-review or review by others with access to the worklist item. Notes 218 may include practical observations regarding a corresponding pathway, treatment plan, or regimen, questions to the creator of the corresponding worklist item, and various other freeform text items.

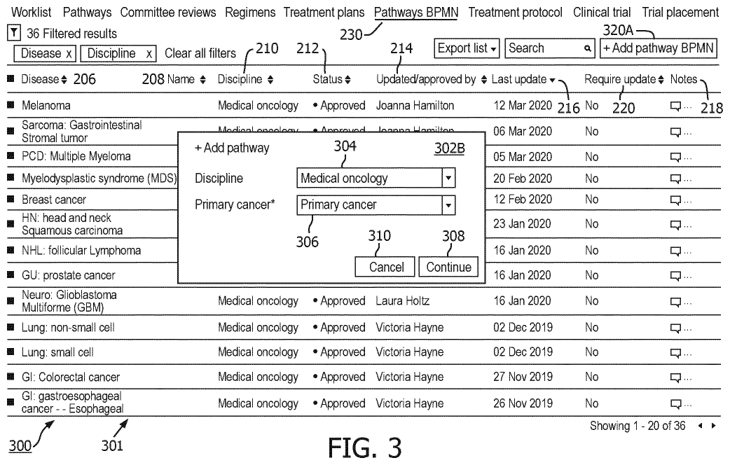

Users may create new pathways 202A by interacting with pathways graph button 230 to enter into a pathways authoring view, such as the example pathways authoring view 300 depicted in FIG. 3. In pathways authoring view 300, a query results 301 displays a list of pathways in tabular, or grid, form.

Query results 301 displays entries with fields for disease 206, name 208, discipline 210, status 212, updated/approved by 214, last update 216, and notes 218, much like as in view 200. According to this view 300, however, only Pathways BPMN items are shown in the list. Further, a field for update requirement 320 is included in query results 301. Update requirement 320 field denotes whether a corresponding pathway item should be updated (e.g., due to an error, new clinical evidence, changing practice, etc.). In some embodiments, the update requirement field 320 may be manually set by one or more users (e.g., based on the user's determination in view of the aforementioned factors) or may be automatically set based on automatic analysis of information related to the pathways. For example, if an automatic validation of the pathway fails, the flag may be set. As another example, if the pathway is linked to a particular piece of clinical evidence (e.g., via an ontological concept or a link to a particular publication stored in the data model) and the system locates a new study impacting that clinical evidence, the update field may be automatically set. Additionally, in such case, the new study may be made available to a user selecting the impacted pathways BPMN or otherwise linked to the data structure of the pathways BPMN.

Moreover, view 300 includes an add pathway button 302A, which users may interact with to create a new pathway and corresponding worklist item. Interacting with add pathway button 302A causes a pathway creation popout 302B to be provided to the user. Pathway creation popout 302B includes a dropdown menus for discipline selection 304 and primary disease 306, from which appropriate options may be selected by a user before creating a new data item in pathways knowledgebase 110 with the selected respective field values. While primary disease 306 is depicted in FIG. 3 as “primary cancer” it is understood that, in some examples, other diseases may be selected and a substantially similar workflow and process may be undergone for creating corresponding pathways, treatment plans, regimen, etc., within the spirit and scope of this disclosure. Once discipline selection 304 and primary disease 306 are set, users may use a graphical computer assisted design (CAD) tool to construct a corresponding pathway as a network node graph, or BPMN tree.

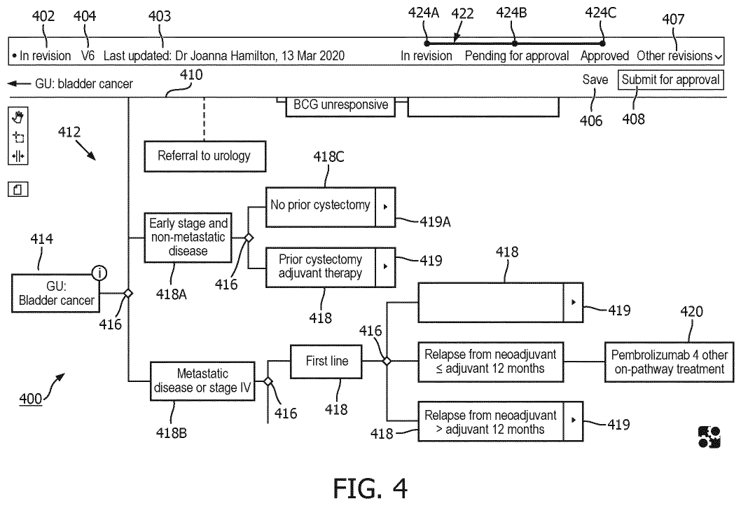

FIG. 4 depicts an example CAD view 400 for constructing a pathway BPMN.

Pathways are displayed on a build area 410 as a graph 412 of decision nodes 418 and terminating treatment nodes 420. A disease node 414 acts as a root node, or starting point, in graph 412 and is automatically generated based on the selected primary disease 306. Here, disease node 414 is labeled as “GU: Bladder Cancer.” In some examples, portions or features of graph 412 may be preconstructed based on primary disease 306 by automatically retrieving related information from pathways knowledgebase 110, such as by generating and connecting certain preconfigured decision nodes 418.

Where multiple decision nodes 418 branch off a preceding decision node 418 or disease node 414, CAD view 400 includes a fork indicator 416 in graph 412 denoting multiple subsequent decision nodes 418. Decision nodes 418 describe specific phenotypes, genotypes, and/or other patient characteristics for navigating through respective branches of graph 412, and thus branches of a corresponding pathway. For example, as depicted in FIG. 4, disease node 414, representing gastrourinary bladder cancer (“GU: Bladder Cancer”), may be either early stage and non-metastatic, as shown by decision node 418a, or metastatic and/or in stage IV, as shown by decision node 418b. Based on which of decision nodes 418a and 418b are selected, different pathway branches can be navigated through to respective terminating treatment node 420.

Some branches may terminate in a pathway launcher 419 rather than a terminating treatment node 420. In such cases, pathway launcher 419 may effectively launch a new pathway through which a user can navigate through as treatment options are explored. For example, as shown in FIG. 4, a decision node 418c may include a patient characteristic of “no prior cystectomy” and, if selected, be associated with a pathway launcher 419a which initiates a new graph 412 representing a corresponding new pathway where a respective disease node 414 includes additional relevant characteristics impacting subsequent decision nodes 418 and terminating treatment nodes 420.

Terminating treatment nodes 420 contain detailed information on treatment options and clinical trials mapped to the respective pathway (e.g., graph 412). The detailed information may be stored in pathways knowledgebase 110. In some examples, the detailed information may additionally include relevant links to external websites or the like, such as government clinical trial repositories (e.g., www.clinicaltrials.gov, www.nih.gov, etc.).

Pathway status is indicated by a progress meter 422, which includes an “in revision” stage indicator 424A, a “pending approval” stage indicator 424B, and an “approved” stage indicator 424C. When a pathway is being drafted or actively edited, in revision indicator 424A is highlighted (e.g., colored green, increased luminosity, increased opacity, etc.). When the pathway has been fully drafted and is awaiting review and approval prior to publication, pending approval indicator 424B is highlighted. Likewise, once the pathway has been approved for release, approved indicator 424C is highlighted. Pathways can be saved, without progressing along a construction workflow of in revision to pending approval to approved, by interacting with a save button 406. In comparison, users may interact with a submit for approval button 408 to conclude drafting of the pathway and submit for reviewer comments and/or approval for publication.

View 400 includes further pathway information. A current stage indicator 402 denotes at which of stage indicators 424A-C the pathway is currently positioned. A version indicator 404 denotes which version of the pathway is currently being viewed. In some examples, pathway version may each be viewed and edited. A last updater field 403 provides identification of the most recent user to update the pathway and the date of the update. Additionally, an “other revisions” tab 407 allows users to access and/or view other revisions of the currently viewed pathway version.

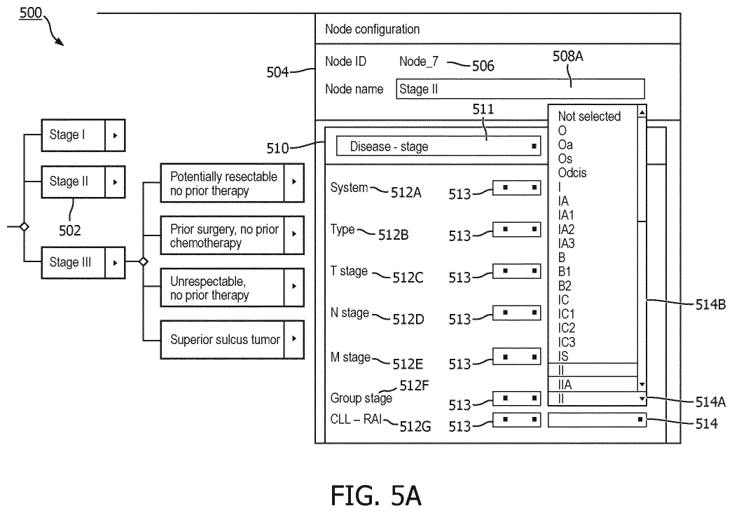

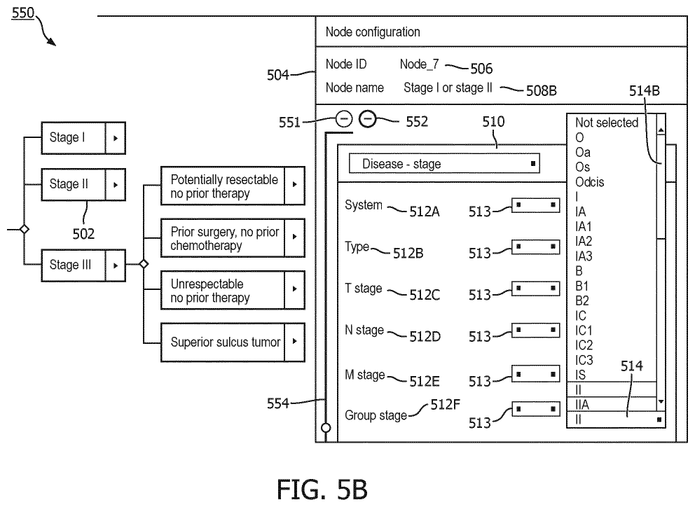

Decision nodes 418 may be individually configured for particular characteristics, phenotypes, genotypes, etc. FIGS. 5A-B depict a node configuration GUI for single characteristic nodes and nodes including Boolean (e.g., “AND,” “OR,” etc.) configurations of characteristics. In FIG. 5A, a zoomed view 500 focuses on a portion of build area 410 including a configuration menu 504 for configuring a decision node 502.

Configuration menu 504 can be used to assign various characteristics to a respective decision node 418. A node ID 506 displays an identifier for decision node 418 being configured that is unique to the respective pathway. In general, node ID 506 is automatically generated by an underlying clinical support system (e.g., by BPMN engine 108, pathways knowledgebase 110, workflow engine 102, etc.). Here, node ID 506 is “Node_7.”

A node name field 508A-B displays an assigned name for the respective decision node 418. In some examples, node name field 508A is automatically assigned a name based on selections in a characteristics submenu 510. In some examples, node name field 508 may be manually assigned a name via user input.

Characteristics submenu 510 includes a collection of characteristic selectors 512A-G based on a selected node type 511. Node type 511 may be selected from a dropdown tab of conditions, patient characteristics, and the like. In some examples, node type 511 may be limited to certain values based on preceding decision nodes 418 and/or a disease node 414. Each characteristic selector 512A-G may include a corresponding dropdown tab 513, which provides a list of corresponding operator values (e.g., “=”, “!=”, etc.) from which a user may choose. Moreover, each characteristic selector 512A-G may include a corresponding characteristic value selector 514, which can be combined with the corresponding operator value to create a positive or negative characteristic requirement.

Here, node type 511 is “Disease-Stage” and characteristic selectors 512A-G include, for example and without imputing limitation, system selector 512A, type selector 512B, T Stage selector 512C, N Stage selector 512D, M Stage selector 512E, Group Stage selector 512F, and CLL-RAI selector 512G. Group Stage selector 512F is associated with an operator value 513 set to “=” and a characteristic value selector 514A set to “II” through an associated characteristic selector dropdown tab 514B, which includes a menu of options based on Group Stage selector 512F. Accordingly, as depicted in FIG. 5A, node 502 is configured to be a Group Stage II node.

In some cases, a decision node 414 may be configured to include a Boolean condition parameter, such as “OR” or “AND” applied to multiple node types 511. FIG. 5B depicts a Boolean node configuration 550 of decision node 502. Node name field 508B is set to “Stage I or Stage II” reflective of an OR conditional 552 being activated in node configuration menu 504. Here, an AND conditional 551 is not activated and so is grayed out, or otherwise visually distinguished from OR conditional 552. A Boolean bracket 554 provides a further visual indicator and grouping of multiple characteristic values set via character value selectors 514.

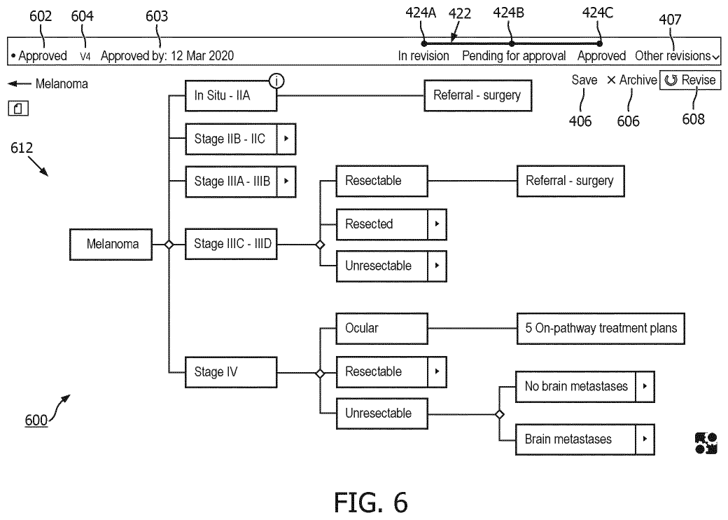

FIG. 6 depicts an example pathway review view 600, where a pathway that has been approved and published may be reviewed via the pathways authoring portal 106. A current stage indicator 602 denotes that the pathway being reviewed has been approved, which corresponds to progress meter 422. Further, a version indicator 604 denotes that the pathway being reviewed is the fourth version (“V4”) and an approver credit 603 identifies the person or entity who approved the pathway being reviewed.

Graph 612 corresponds to the pathway being reviewed and may be navigated through as described above. Moreover, progress through graph 612 may be saved and revisited at a later time by interacting with save button 406. Should a user wish to archive (e.g., place into an inactive storage state, etc.), the user may interact with an archive button 606 provided via the GUI.

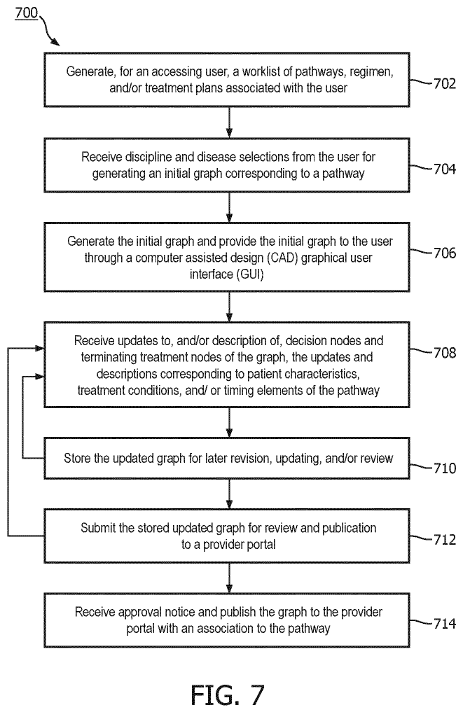

FIG. 7 depicts an example method 700 for creating and modifying a pathway through, for example, pathways authoring portal 106. Users may perform method 700 through a system substantially similarly architected as system architecture 100 described above and depicted in FIG. 1.

At operation 702, a worklist of pathways, regimen, and/or treatment plans associated with an accessing user is generated and displayed, for example, though a GUI to the accessing user. For example, the user may be a clinician authorized by their institution to create pathways through pathways authoring tool 106. When reviewing the worklist through a respective account, the user may be presented with a listing of pathways, regimen, and/or treatment plans upon which they worked or which are associated with their institution and currently under construction or revision.

At operation 704, the user provides selection of a discipline (e.g., medical oncology, radiation oncology, etc.) and a disease to, for example, pathways authoring portal 106 for generating an initial graph corresponding to a pathway. The initial graph may include a root node, such as disease node 414, based on the disease selection. In some examples, additional nodes, such as decisions nodes 418 and or terminating treatment nodes 420 may be automatically generated in whole or in part based on the disease selection, the discipline selection, variables associated with the user or user institution, or based on additional interfacing internal and/or external services (e.g., via API, microservice mesh, etc.).

At operation 706, the initial graph is generated and provided to the user through a computer assisted design (CAD) graphical user interface (GUI). Elements of the graph can be mapped to data stored in a BPMN database and/or a knowledgebase storing various relevant information for pathways, regimen, and treatment plans. In some embodiments, the initial graph may be generated as an empty graph (but with appropriate data structures established for receiving new node definitions), as a single node graph (e.g., including only a root node), or as a multi-node graph (e.g., importing an existing graph or graph template to be further modified).

At operation 708, updates and/or descriptions (e.g., configuration settings) are received (e.g., by pathways authoring tool 106, etc.) for decision nodes and/or terminating treatment nodes of the initial graph. The updates and descriptions may correspond to patient characteristics, treatment conditions, and/or timing elements of the pathway represented by the graph.

At operation 710, the updated graph is stored for later revision, updating, and/or review. The graph, and respective mapping information to data in a knowledgebase and/or BPMN network, may be stored in a local database, cloud storage, remote store or repository, etc. As depicted, the user may later access the stored updated graph to further modify or revise the graph.

At operation 712, the stored and updated graph is submitted for review and, pending approval, publication to a provider portal, such as pathways provider portal 104. The graph may be reviewed by an authorized reviewer, such as, for example, a manager, peer, practice head, review committee, peer group, etc. Where review results in a call for further revisions, the user may be notified and can further revise the graph as needed. In some examples, the reviewer(s) may include comments, guidance, suggestions, and the like along with a requirement that the graph be further revised. Accordingly, as depicted in FIG. 7, method 700 may loop back to operation 708 or proceed to operation 714, as is appropriate.

At operation 714, notice of the approval is received and the graph is published to the provider portal and associated with the corresponding pathway. As a result, clinicians, physicians, providers, and other authorized users of the provider portal may access and deploy the graph to support respective usage of the corresponding pathway.

Returning to FIG. 1, pathways provider portal 104 receives inputs for accessing, managing, selecting, navigating, and executing published pathways. In addition, users of the clinical support system may access a patient dashboard providing aggregated clinical and genomic data to support selecting and/or navigating an appropriate pathway. In some examples, pathways provider portal 104 may automatically recommend pathways, regimen, and/or treatment plans or therapies based on the aggregated data provided by the patient dashboard.

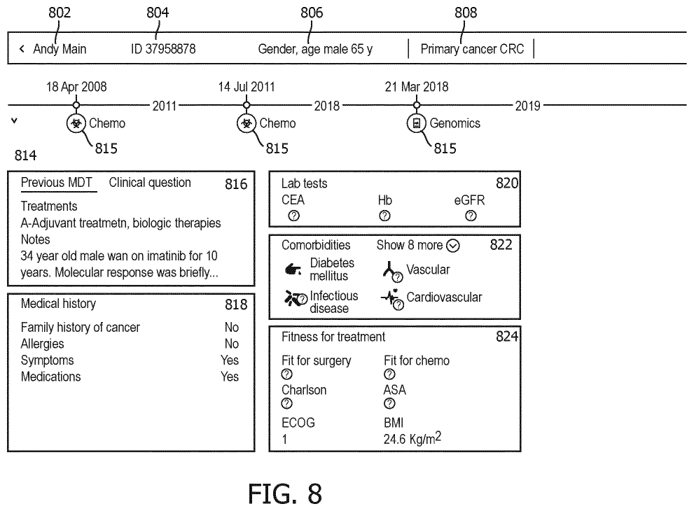

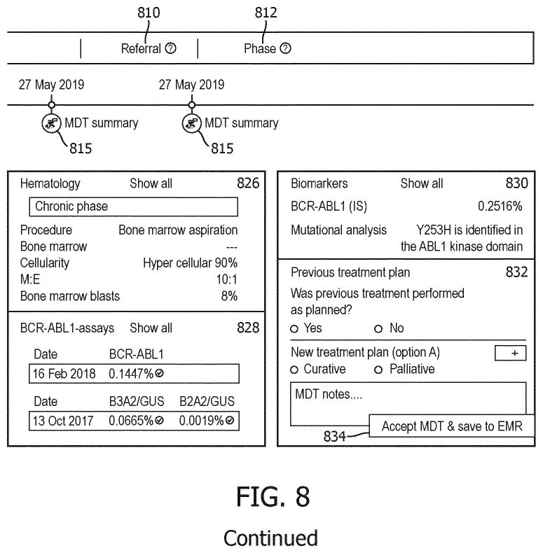

FIG. 8 depicts a patient dashboard 800 for reviewing patient data through an intuitive and informative GUI. Patient dashboard 800 includes both basic patient information, such as identity, gender, age, primary cancer, etc., as well as detailed report tiles 816-832 each including important medical information for making clinical decisions throughout treatment of the patient.

The patient is identified by a name field 802 and an ID value 804. A gender and age field 806 display the gender and age of the patient, and a primary cancer field 808 displays a disease or condition for which the patient is being treat. While FIG. 800 depicts a patient undergoing treatment for colorectal cancer (“CRC”), it will be understood by a person having ordinary skill in the art that other diseases and/or conditions may be treat with the aid of the contents of this disclosure, including, but without imputing limitation, various cancers and other patient conditions.

Further, patient dashboard 800 includes a referral link 810, for identifying from whom the patient referral came from and whether it was internal or external to the treating organization, and phase identifier 812, which may display which phase of a disease the patient is currently in (e.g., first phase, chronic phase, recovery phase, etc.). A treatment history timeline 814 provides a reviewing user a summary of treatments and tests the patient has undergone over multiple years. Treatment event icons 815 indicate when the patient underwent a treatment or test relative to each other and with a precise date. For example, treatment history timeline 814 includes treatment event icons for chemotherapy (“chemo”), genomics testing, and multidisciplinary treatments (MDTs).

Detailed report tiles 816-832 are individual report windows and, in some examples, may be customized according to a user's preferences (e.g., size, data, visual properties, etc.). Here, detailed report tile 816 provides a scrollable summary of previous MDTs and a tab for reviewing a corresponding clinical question. Detailed report tile 818 provides a summary medical history for the patient, such as relevant family history, allergies, symptoms, medications, etc.

Detailed report tile 820 provides lab test information such as carcinoembryonic antigen (CEA) test information, hemoglobin blood (Hb) test information, and estimated glomerular filtration rate (eGFR) test information. Detailed report tile 822 includes comorbidities information (e.g., diabetes, infectious disease, vascular, cardiovascular, etc.). Detailed report tile 824 includes an estimate, and related or supporting information, for the patient's fitness for treatment, and may include specific treatments such as surgery, chemo, etc. Detailed report tile 826 includes hematology information and detailed report tile 828 includes assays information. Biomarker information is provided in detailed report tile 830. Detailed report tile 832 includes interactable response items for which the user may provide input related to previous treatment of the patient. For example, in patient dashboard 800, the user may set whether a previous treatment performed as planned and whether a new treatment plan is curative or palliative via radio icons in detailed report tile 832. Once the user has input appropriate information in detailed report tile 832, a commit button 834 may be pressed to approve the information and enter it (e.g., via API call, etc.) into a respective electronic medical record (EMR) for the patient.

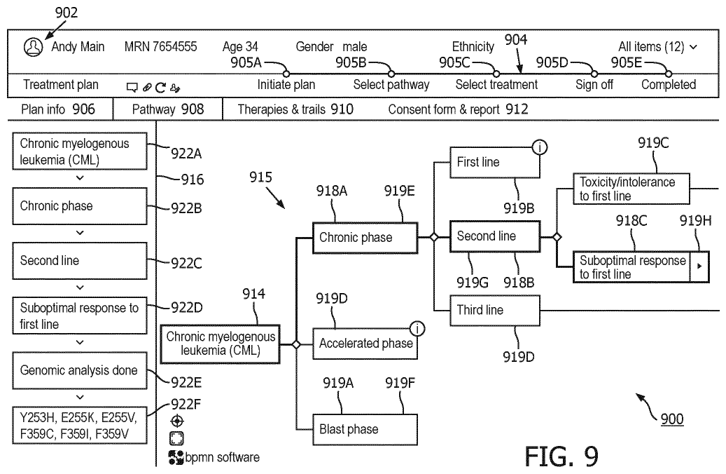

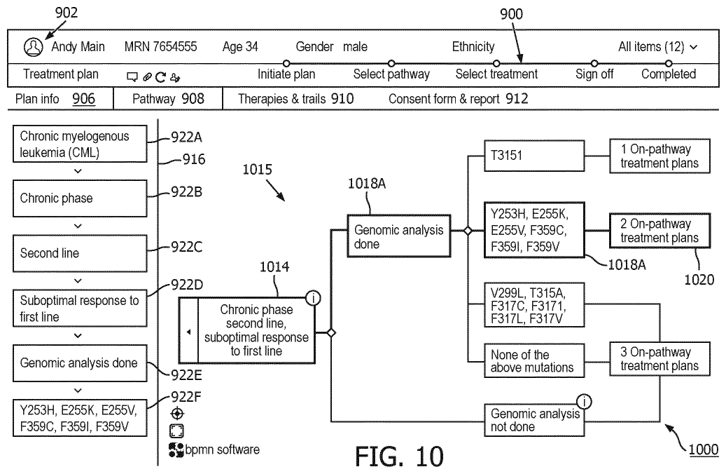

FIGS. 9 and 10 depict example clinical pathway navigation views 900 and 1000 based on information from patient dashboard 800. Here, biomarker descriptions are used to represent forking events in treatment plans and/or pathways. A clinical pathway on chronic myelogenous leukemia may stratify on first line compared to second line therapy, such as on ABL1 sensitizing mutations (e.g., Y253H). Based on this information in the corresponding pathway BPMN representation and patient profile representation, the BPMN engine performs matching and automatically generates a suggestion presented over the pathways provider portal GUI.

Clinical pathway navigation view 900 is a view of an initial treatment for a patient with chronic myelogenous leukemia. Clinical pathway navigation view 900 includes a patient information summary bar 902 including patient information such as name, identifier, age, gender, ethnicity, etc., that may be useful in selecting steps within a clinical pathway. A workflow meter 904 includes steps within a treatment workflow, such as indicators for “initiate a plan” 905A, “select pathway” 905B, “select treatment” 905C, “sign off” 905D, and “completed” 905E. Moreover, tabs 906-912 can be accessed through clinical pathway navigation view 900, including plan info tab 906 for viewing treatment plan information, pathway tab 908 for navigating and reviewing a clinical pathway, therapies and trials tab 910 for reviewing therapy and trial options available to the patient based on patient information and related pathway progress, and a consent form and report tab 912 for reviewing, retrieving, and completing consent forms and reports needed for participating in clinical trials, therapies, tests, and the like.

In some examples, navigation between nodes is performed automatically by processing a corresponding patient EMR. Data is retrieved from the patient EMR and checked against conditional values built into each node at pathway creation as described above. In some examples, the patient EMR may first be converted into an appropriate data structure such as, for example and without imputing limitation, an XML, or JSON object. A node may then include an operation to automatically retrieve a patient characteristic from the converted object through, for example, a “getter” call directly to the object. For example, a node may validate a patient's age before automatically navigating to the appropriate node through a “patient.age( )” value entry, where “patient” identifies a data object converted from an EMR. In some examples, a dictionary or ontology, stores mappings for node fields, or conditions, and various functions or data retrieval calls (e.g., getters, etc.). In some examples, which dictionary or ontology is used may depend on institution, department, disease, and/or the user who authored the pathway.

Here, the patient is non-responsive to first line therapy and therefore is matched to second line therapy and particular mutations on the ABL1 gene. Decision nodes 919A-H extend from disease node 914 and display a visual path through graph 915 reflecting selected steps 918A, 918B, and 918C of a corresponding pathway.

A history pane 916 shows all selected steps 922A-F corresponding to a respective pathway arm that has been walked through as appropriate for the current patient at hand. In particular, history pane 916 displays steps for steps through graph 915 and also steps through a subsequent graph representing a related subsequent clinical pathway undergone by the patient after reaching terminal decision node 918C representing the patient exhibiting a suboptimal response to first line therapy. In various embodiments, the system may generate the content of the history pane 916 dynamically based on the underlying data depicted by the graph 915.

In some examples, the graph 915 is stored as an XML file in a database. Navigated paths through the graph 915 (e.g., the respective pathway arm corresponding to steps 922A-F) may be saved as a separate data object storing a business process execution, which may be retrieved and interpreted for rendering by a BPM engine. The business process execution data object stores references to items or entries within a corresponding XML file describing a pathway graph and can be accessed to generate a navigation of a pathway and/or content of the history pane 916. The corresponding XML file is version controlled and, as a result, process execution objects each reference a particular version of a pathway, the other versions of which may be stored as independent XML files or, in some examples, as one or more iterations (e.g., differences) applied to a shared underlying (e.g., version 1.0) XML file.

For example, where the system stores a pathway as a tree data structure (e.g., an XML data object), the system may create a new summary data structure (e.g., an ordered list, a tree, or another business process execution format), copy the root node of the pathway tree data structure to the summary data structure, traverse to the next node on the pathway tree data structure based on patient data (e.g., patient data indicating the next node taken or patient data that can be interpreted in conjunction with the pathway tree data structure to infer a next node), copy that next node to the summary data structure, and continue in this manner until the entire patient journey to date has been captured in the summary data structure. The system may then translate the summary data structure into a graphical representation such as the linear flow-chart shown in the history pane 916 or another suitable alternative representation to be included in the history pane 916.

While the history pane 916 is shown together on the same view 900 as the full graph 915, it will be appreciated that the history pane 916 may be shown without the full graph 915 on other views as a summary of the patient's treatment path to date. For example, in some embodiments, when the user selects the therapies & trials tab, a different view (not shown) may be displayed that includes information related to available therapies and trials for the patient along with the history pane 916.

Clinical pathway navigation view 1000, depicted in FIG. 10, reflects steps 922E and following and displays a graph 1015 corresponding to a clinical pathway reflective of the earlier pathway. In comparison to pathway navigation view 900, a root node 1014 is not directly related to a disease (e.g., chronic myelogenous leukemia) but instead to the earlier pathway arm that has been walked through in treating the current patient at hand. As a result, root node 1014 includes a summary of navigation through graph 915, including “chronic phase, second line, suboptimal response to first line.” Decision nodes 1018A and 1018B are likewise reflected in history pane 916 as selected steps 922E and 922F. A terminal treatment node 1020 provides on-pathway treatment plans which are suggested according to the preceding nodes throughout both graphs 915 and 1015.

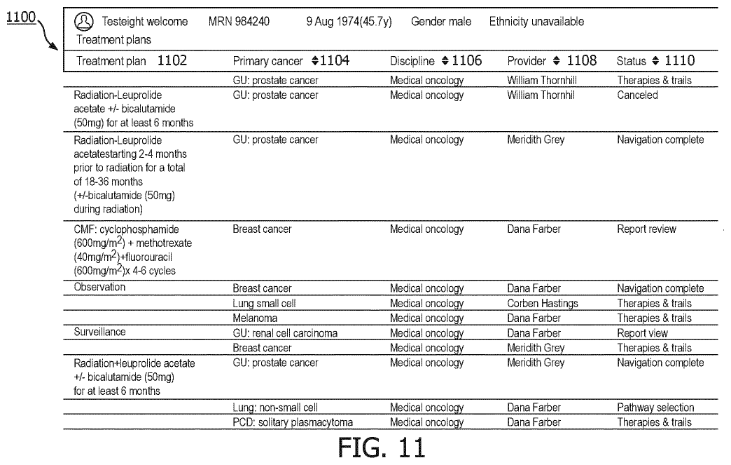

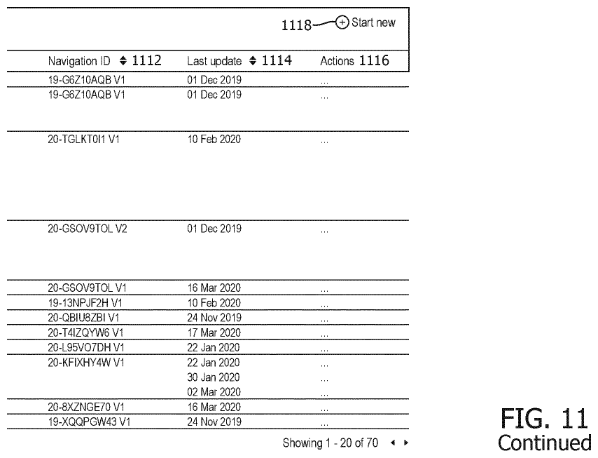

FIG. 11 depicts an example treatment plan selection view 1100 for accessing treatment plans from among those available to the user. Treatment plans are presented to the user in a tabular format and in a summarized fashion. Individual treatment plans are each shown as row entries.

Each row includes a treatment plan name 1102, if available, a primary cancer 1104 for which the treatment plan is intended, a discipline 1106 (e.g., medical oncology, radiation oncology, etc.), a provider name 1108 who has submitted the treatment plan, a status 1100, a navigation identifier 1112, a last update 1114, and an actions menu 1116 which may be accessed to perform select actions. Further, the user may initiate a new treatment and pathway navigation by interacting with a “start new” button 1118.

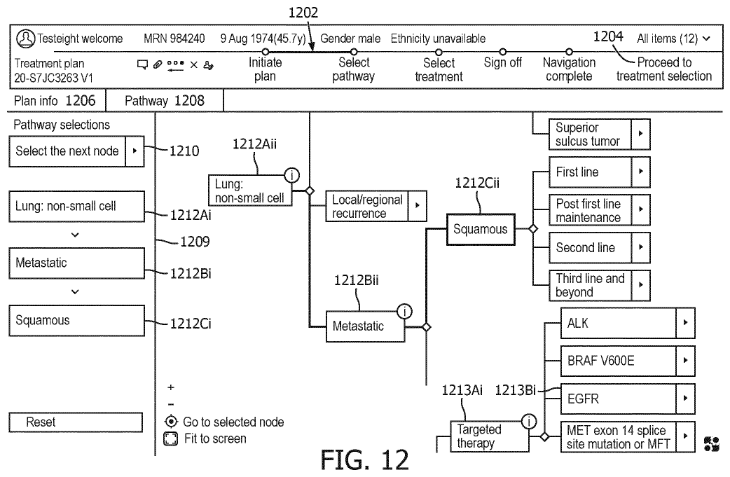

FIG. 12 depicts an example pathway navigation view 1200 similarly to FIGS. 9-10. Here, a pathway is initiated following selection of a plan from treatment plan selection view 1100. A workflow meter 1202 provides a graphical representation to the user of the completed steps along a corresponding treatment workflow.

Plan info tab 1206 and pathway tab 1208 are made available to the user in pathway navigation view 1200. As additional steps are completed, as shown in workflow meter 1202, additional tabs may be made available to the user.

Graph 1215 corresponds to the selected pathway and, as graph 1215 is navigated, history pane 1209 populates with corresponding pathway selections. Further, a node selection tab 1210 allows the user to navigate graph 1215 through a dropdown tab of available options rather than, or in addition to, clicking on nodes within graph 1215. A pathway selection 1212Ai corresponds to a node 1212Aii for “lung non-small cell,” a pathway selection 1212Bi corresponds to a node 1212Bii for “metastatic,” and a pathway selection 1212Ci corresponds to a node 1212Cii for squamous. Each of the selections and corresponding nodes provide further characteristic information of a disease (e.g., lung cancer, etc.) being treated through the selected pathway. Additional nodes not selected here include a “targeted therapy” node 1213Ai and subsequent “EGFR” node 1213Bi.

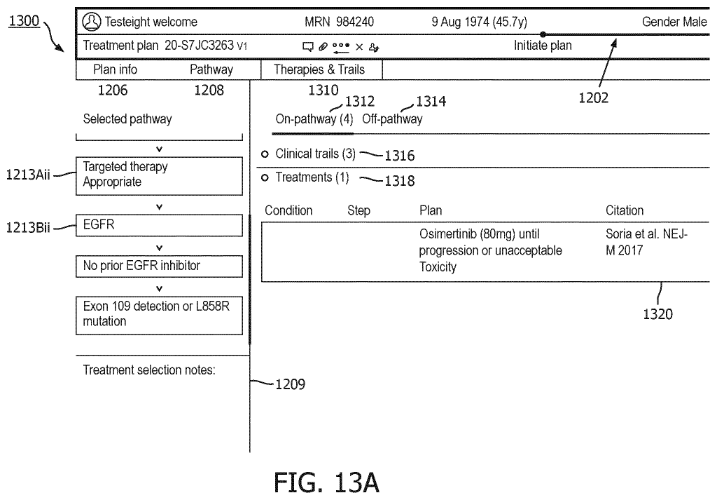

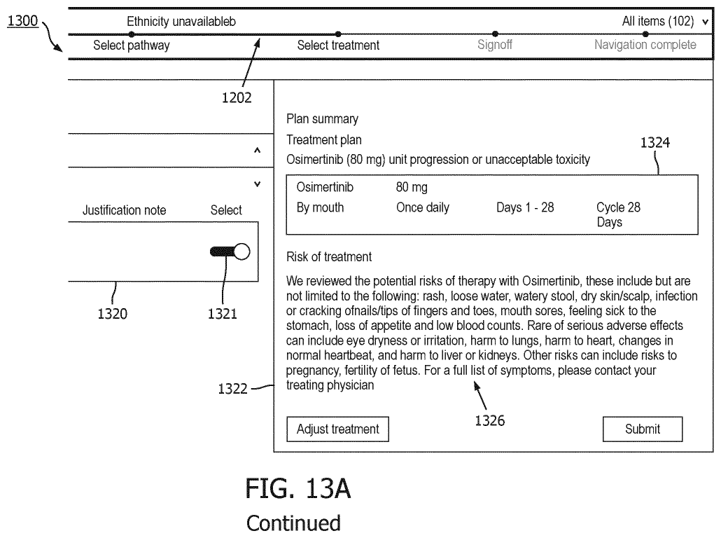

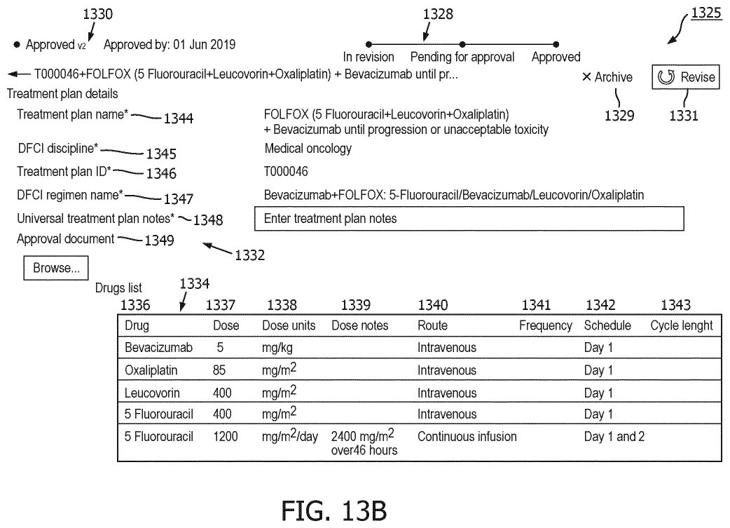

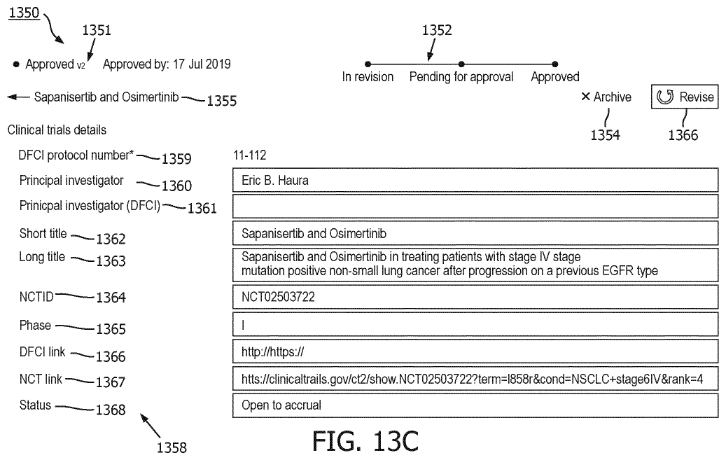

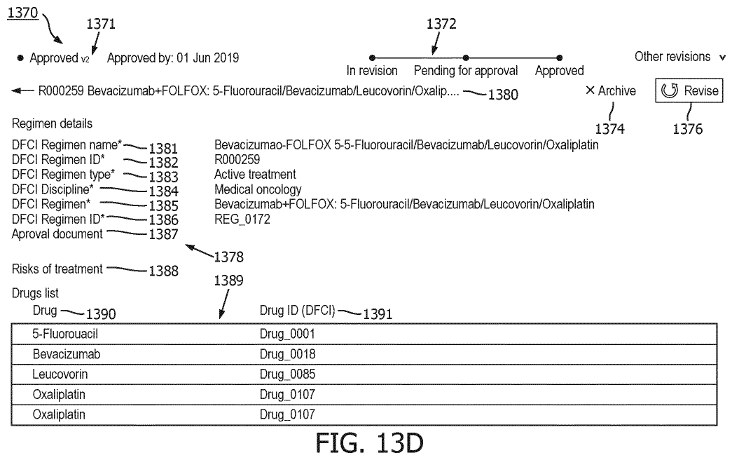

FIG. 13 depicts a therapies and clinical trials view 1300 in which targeted therapy node 1213Ai and EGFR node 1213Bi have been selected and therapies and trials have been accordingly matched for the patient. For example, one or more nodes of a patient's particular pathway (e.g., the final node selected for the patient on the full pathway or nodes 1213Aii, 1213Bii) may include identifiers for one or more treatments or clinical trials. The system may read the data structure corresponding to each node on the patient's particular selected nodes within the pathway, gather any identifiers for clinical trials or treatments, retrieve further information describing these trials or treatments (e.g., by querying the identifiers in a database), and display the information in an organized and interactive format via the therapies and clinical trials view 1300. Pathway selections 1213Aii and 1213Bii corresponding to nodes 1213Ai and 1213Bi have been added to history pane 1209, thereby presenting a summary view of the patient's particular path taken through the full pathway. In particular, workflow meter 1202 has progressed to “select treatment” and a therapies and trials tab 1310 has been made available to the user.

Upon accessing therapies and trials tab 1310, subtabs are made available to the user for reviewing and/or selectin on and off pathway treatments through an on-pathway subtab 1312 and an off-pathway subtab 1314. On-pathway subtab 1312 displays clinical trials 1316 and treatments 1318 that are matched to the pathway navigation above by the clinical support system. In contrast, off-pathway subtab 1314 allows a user to manually select a treatment or clinical trial which the clinical support system did not automatically suggest.

Here, treatments 1318 include a first treatment plan 1320 that has been selected via selection toggle 1321. A plan summary pane 1322 is provided for summarized information of the selected treatment plan. Plan summary pane 1322 includes a regimen 1324, which includes a drug dosage, drug intake method (e.g., “by mouth”), frequency (e.g., “once daily”), duration (e.g., “days 1-28”), and scheduling information (e.g., “cycle 28 days”). Moreover, a risks description 1326 provides a risk assessment of the treatment in descriptive, narrative (e.g., unstructured string text, etc.) terms. The user may adjust or confirm the treatment through plan summary pane 1322.

FIG. 13B depicts a treatment plan view 1325 which may be accessed through the pathways provider portal and/or modified through either the pathway authoring portal or by adjusting a treatment through a plan summary pane 1322, discussed above in reference to FIG. 13A. Treatment plan view 1325 includes version information 1330, denoting an approval status, version, and, in the case of an approved treatment plan, an approval date or, in the case of a treatment plan under revision, a time, date, and author of most recent edits to the treatment plan under revision. Furthermore, a workflow meter 1328 denotes which step of a revision workflow the treatment plan is in, such as “in revision,” “pending for approval,” and “approved.” Here, an approved treatment plan is shown and treatment plan view 1325 includes an archive button 1329 and a revise button 1331 for deleting and setting the treatment plan to “in revision,” respectively.

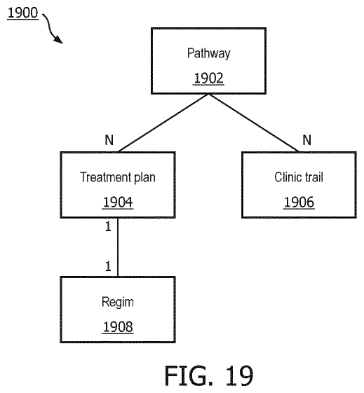

Treatment plan view 1325 includes a treatment plan details section 1332 which provides users with various details of the treatment plan for determining whether the treatment plan is appropriate for a patient. Treatment plan details section 1332 includes treatment plan name 1344, discipline 1345, treatment plan identifier 1346, regimen name 1347, treatment plan notes 1348, and an approval document 1349. Discipline 1345 may be based on institutional definitions or practice. Here, discipline 1345 is according to Dana-Farber Cancer Institute (DFCI) definitions and is set to “Medical Oncology.” Treatment plan identifier 1346 is an alphanumeric identifier for the treatment plan. Regimen name 1347 denotes the name of a regimen associated with the treatment plan, further discussed below in reference to FIG. 13D and FIG. 19. Treatment plan notes 1348 includes a free form text field for storing notes regarding the treatment plan that may be viewed by other users accessing the treatment plan through the clinical support system. Approval document 1349 includes an approval document attachment (e.g., PDF, DOC, JPG, etc.) documenting and/or verifying an associated approval process.

Further, treatment plan view 1325 includes a drug list 1334 and information related to an associated drug regimen. Drug list 1334 includes a tabular list of drugs provided as rows. Each drug (row) includes a drug name 1336, a dose 1337, a dose units 1338, dose notes 1339, dose frequency 1341, dose schedule 1342, and cycle length 1343. In effect, a user may view key drug regimen information associated with a treatment plan in a single, accessible interface to determine appropriateness of the treatment plan for treating a patient.

FIG. 13C depicts a clinical trial view 1350 which may be accessed, for example, through therapies and clinical trials view 300 discussed above. In general, each clinical trial listed in therapies and clinical trials view 300 may include a link to a respective clinical trial view 1350 for a user to review important clinical trial summary information in determining appropriateness of the clinical trial for treating a patient. Clinical trial view 1350 includes version information 1351, denoting an approval status, version, and, in the case of an approved clinical trial description, an approval date or, in the case of a clinical trial description under revision, a time, date, and author of most recent edits. Furthermore, a workflow meter 1352 denotes which step of a revision workflow the clinical trial description is in, such as “in revision,” “pending for approval,” and “approved.” Here, an approved clinical trial description is shown and so clinical trial view 1350 includes an archive button 1354 and a revise button 1356 for deleting and setting the clinical trial description to “in revision,” respectively.

A clinical trial name 1355 is displayed above a clinical trial details pane 1358. Clinical trial details pane 1358 includes a protocol number 1359, principal investigator name 1360 and institute-linked principal investigator name 1361, a short title 1362, a long title 1363, a repository-based clinical trials identifier 1364, a phase 1365, an institute link 1366, a clinical trial repository link 1367, and a status 1368. In general, clinical trial name 1355 and short title 1362 are the same, whereas long title 1363 includes additional descriptive terms for the clinical trial. Repository-based clinical trials identifier 1364 may, for example and without imputing limitation, be an associated identifier for accessing the clinical trial on www.ClinicalTrials.gov or some other official repository of clinical trials. Likewise, clinical trial repository link 1367 may include a direct hyperlink to a respective clinical trial page on the official repository, such as www.ClinicalTrials.gov. Where a clinical trial is affiliated with a particular institution and the affiliated institution has associated web content available, institute link 1366 may include a link to the associated web content. Phase 1365 denotes which phase the clinical trial is in and status 1368 denotes a subject status, such as “OPEN TO ACCRUAL,” of the clinical trial.

FIG. 13D depicts a regimen view 1370 which may be accessed via the clinicals support system for reviewing a regimen associated with a particular treatment plan or the like. Regimen view 1370 includes version information 1371, denoting an approval status, version, and, in the case of an approved regimen, an approval date or, in the case of a regimen under revision, a time, date, and author of most recent edits to the regimen under revision. Furthermore, a workflow meter 172 denotes which step of a revision workflow the regimen is in, such as “in revision,” “pending for approval,” and “approved.” Here, an approved regimen is shown and regimen view 1370 includes an archive button 1374 and a revise button 1376 for deleting and setting the regimen to “in revision,” respectively.

Regimen view 1370 includes a regimen name 1380 displayed above a regimen details pane 1378 and a drug list pane 1389. Regimen details pane 1378 provides various user details and drug list pane 1389 provides a detailed listing of drugs included in the regimen.

Regimen details pane 1378 includes a regimen name 1381, regimen identifier 1382, regimen type 1383, discipline 1384, secondary regimen name 1385, secondary regimen identifier 1386, approval document 1387, and treatment risks description 1388, each of which may be structured or formatted according to institution-specific practice. In cases where a regimen may have more than one name or identifier, secondary regimen name 1385 and secondary regimen identifier 1386 may denote the additional name and identifier, respectively. Approval document 1387 may include an attachment providing documentation and/or verification that the regimen has been approved for use. Treatment risks description 1388 can include free form text description of risks associated with the regimen.

Drug list pane 1389 displays a tabular listing of drugs used in the regimen, with one drug per row. Each drug (row) includes a drug name 1390 and a drug identifier 1391. Drug identifier 1391 is an alphanumeric identifier for looking the drug up in systems and databases.

In general, each regimen described by regimen view 1370 is associated with a single treatment plan (e.g., as described by treatment plan view 1350 discussed above).

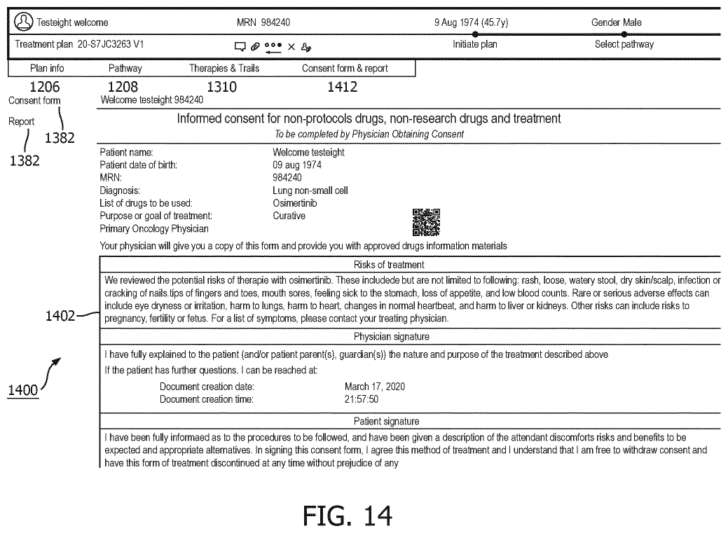

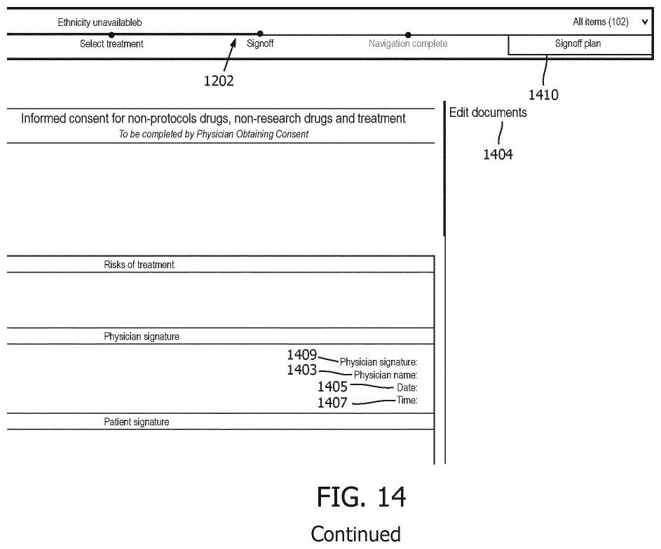

FIG. 14 depicts a consent forms and report view 1400 for reviewing and retrieving consent forms and reports associated with the selected treatment plan. A consent forms and report tab 1412 allows for the user to navigate to consent forms and report view 1400. The clinical support system retrieves appropriate consent forms and generates appropriate reports based on selected therapies and/or trials. Here, an informed consent for non-protocol drugs, non-research drugs and treatment form 1402 has been provided by the clinical support system for signing and/or printing.

Consent forms and report view 1400 provides both consent forms (e.g., form 1402) and reports, as appropriate. Consent forms and reports may be respectively accessed via a consent form link 1406 and a report link 1408. Additionally, users may manually edit documents directly within consent forms and report view 1400 by toggling an edit documents toggle 1404. When edit documents toggle 1404 is activated, the user can enter information in appropriate fields of an editable consent form PDF, such as a signature field 1409, a physician name field 1403, a signature date field 1405, and/or a signature time field 1407.

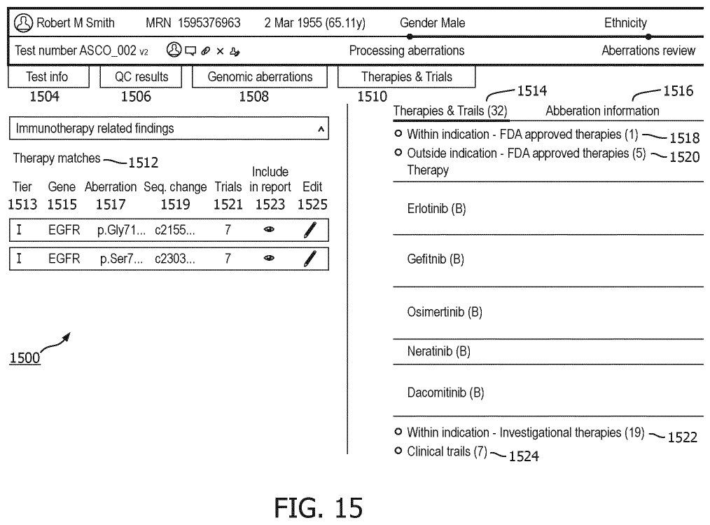

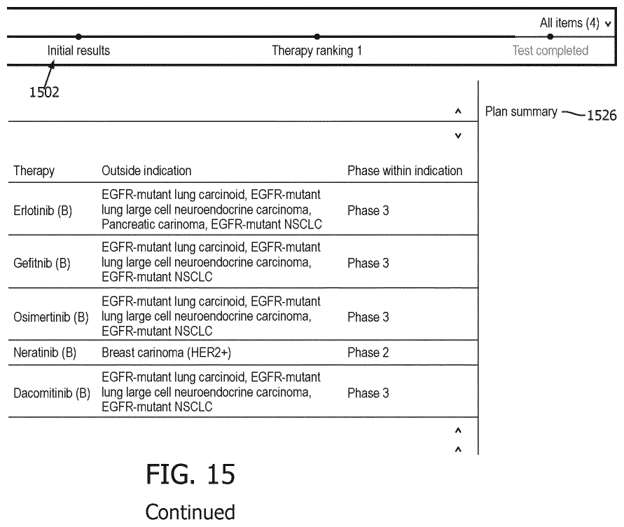

FIG. 15 depicts another example of a therapies and trials view 1500 that, in addition to aspects discussed above in reference to FIG. 13, includes an automatic therapy matching output. Where therapies and trials view 1300 is accessed through pathways navigation, therapies and trials view 1500 may be accessed through testing workflows provided by the clinical support system and managed by workflow engine 102. In particular, a genomic test 1530 is associated with a workflow represented by workflow meter 1502, which tracks aberrations processing, aberration review, initial results, therapy ranking, and test completed steps in sequence.

In addition to therapies and trials 1514 and aberration information 1516 within therapies and trials tab 1510, contextual and supporting information can be accessed through additional tabs of therapies and trials view 1500. Genomic test information can be accessed through a test info tab 1504. Quality control information can be accessed through a QC results tab 1506. Genomic aberrations information can be accessed through a genomic aberrations tab 1508.

Therapies and trials 1514 provides a list of therapies and/or clinical trials relevant to the subject of the genomic test. FDA approved therapies for which the testing results are within indications is provided through a within indication—FDA approved therapies list 1518. An outside indication—FDA approved therapies list 1520 provides a list of FDA approved therapies for which the testing results are not within indications. Investigational therapies for which the testing results are within indication are listed in a within indication—investigational therapies list 1522. Clinical trials list 1524 provides a list of clinical trials in which the patient may be qualified to participate. A plan summary pane 1526 provides summary information similarly to as discussed above when a listed item is selected from therapies and trials tab 1514.

A therapy matches pane 1512 presents the user with therapies matched to the genomics test and/or other characteristics of the patient or respective treatment history. Therapy matches are described in therapy matches pane 1512 by respective tier 1513, relevant gene match 1515, relevant aberration match 1517, relevant sequence change 1519, and number of trials 1521. Further, each match includes a report inclusion toggle 1523 and an edit toggle 1525. The user may interact with report inclusion toggle 1523 to include the respective therapy match in generated reports. The user may interact with edit toggle 1525 to manually modify the therapy match information.

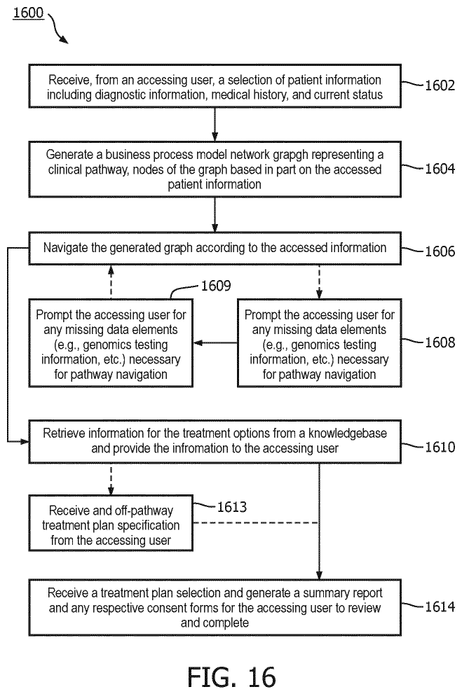

FIG. 16 is a flowchart of a method 1600 for managing treatment of a patient using clinical pathways. At operation 1602, a selection of patient information including patient diagnostic information, medical history, and current status is received from an accessing user. For example, the user may select a patient identifier from a listing and the system may automatically retrieve patient information or be directed to the patient information for automatic retrieval via user inputs.

At operation 1604, a BPMN graph is generated that represents a clinical pathway where nodes of the graph are based in part on the accessed patient information. The BPMN graph can be generated by, for example, BPMN engine 108 and elements of the BPMN graph, such as an underlying pathway, node configurations, rules, etc., can be retrieved from a knowledgebase. In some examples, the BPMN graph may be based on a graph produced through pathways authoring portal 106, as discussed above.

At operation 1606, the generated graph is navigated, in some examples automatically, according to the accessed information. For example, BPMN engine 108 and workflow engine 102 may retrieve information from the accessed patient information and automatically navigate through decision nodes of the generated graph. Alternatively, or additionally, a user may manually navigate some or all of the nodes of the generated graph. For example, a user may directly indicate one or more nodes to add to the current patient's path through the pathway. Alternatively, in some embodiments, the BPMN engine 108 or workflow engine 102 may query the user by requesting a selection of a next node or, as will be explained in greater detail with respect to operation 1608, requesting input of additional patient information, which will be used by the BPMN engine 108 and workflow engine 102 to make an automatic selection of one or more node.

At operation 1608, the accessing user is prompted for any missing data elements (e.g., genomics testing information, etc.) necessary for navigating the graph. At operation 1609, user inputs are received responsive to the prompt and navigation continues. As a result, operations 1606-1609 may repeat as needed. In some examples, inputting missing data elements responsive to prompts may bring the accessing user to a different view or tab (e.g., FIG. 15 discussed above, etc.) associated with retrieving the missing data elements. In the case of a genomics test, the accessing user may save progress through the graph and return to input the missing data elements at a later time following respective testing and the like.

At operation 1610, navigation reaches a terminal node of the graph which represents treatment options based on a path through the graph corresponding to the preceding navigation. It will be appreciated that, while some embodiments may place treatment options only on terminal nodes, other embodiments may enable treatment options to be attached to intermediate nodes of the pathway. In such embodiments, the method 1600 may be paused at operations 1606, 1608, or 1609 to display intermediate therapy options. In some such embodiments, if the user selects the proceed to treatment selection button 1204 of FIG. 1200 before a terminal node has been selected, the system may nonetheless display the therapies and trials view 1300 of FIG. 13A based on the available information. For example, the system may identify any therapies and clinical trials identified by the non-terminal nodes that have been selected for the patient and display them on the therapies and trials view 1300. Such non-terminal nodes may, for example, identify treatments for particular symptoms that are acceptable to administer before the pathway is completed. Alternatively, or additionally, the system may identify which terminal nodes might still be reached from the patient's current position in the pathway (e.g., by pruning the pathway tree data structure of any paths that diverge from the patient's current path and subsequently reading all remaining leaf nodes). From the possible terminal nodes, the system may identify all possible clinical trials and treatments, and display corresponding descriptive information on the therapies and trials view 1310. Such display may include, for each therapy and trial, an indication that the recommendation is preliminary in nature and that the patient's pathway has not been completed. It will also be appreciated that intermediate nodes may identify procedures other than treatments. For example, an intermediate node may identify diagnostic procedures to be ordered which may help advance that patient's journey through the pathway. In some such embodiments, the therapies and trials view 1300 or another view (not shown) may provide a list of diagnostic procedures or other recommended tasks identified by the nodes on the patient's path through the pathway.

At operation 1612, information for the treatment options is retrieved from a knowledgebase and provided to the accessing user. Optionally, at operation 1613, an off-pathway treatment plan specification is received from the accessing user. For example, the accessing user may be brought to therapies and trials view 1300 discussed above for selecting on-pathway treatment plans or providing an off-pathway treatment plan selection and reasoning.

At operation 1614, a treatment plan selection is received and a summary report and any respective consent forms are generated for the accessing user to review and complete. For example, the accessing user may be provided with view 1400 discussed above. Consent forms and related documents may be edited and modified and then printed out for patient signature. In some examples, the forms and documents can be provided to a downstream process to automatically distribute forms and solicit signing from the patient, either electronically or as a physical signature.

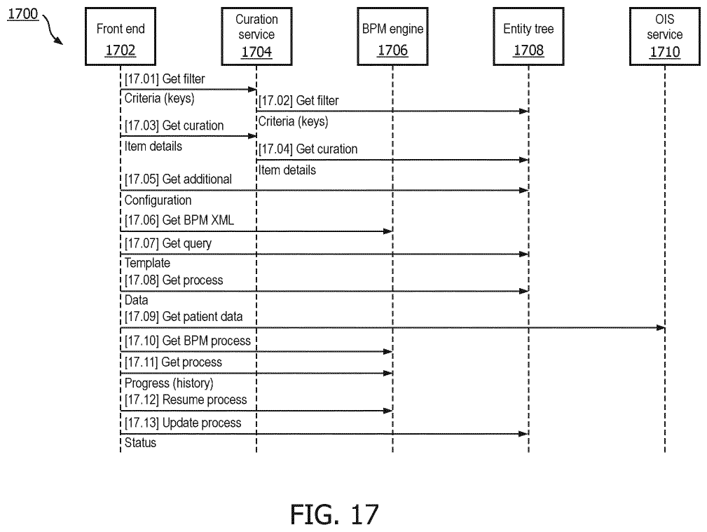

FIG. 17 is a sequence diagram 1700 for running and matching decision trees (BPMN graphs). The message sequence of sequence diagram 1700 interchanges across a front end 1702, a curation service 1704, a BPMN engine 1706, an entity tree 1708, and an oncology information system (OIS) services 1710. A user utilizes front end 1702 and, from the perspective of the user, front end 1702 initiates and drives the message sequence.

Front end 1702 first transmits to curation services 1704 a message 17.01 including filter criteria and access keys. Curation services 1704 then interacts with entity tree 1706 via process 17.02 including forwarding a retrieval command (e.g., a GET command) with the filter criteria and access keys of message 17.01.

Front end 1702 then interacts with curation services 1704 to perform a process 17.03 for retrieving curation item details (e.g., a GET command). In response, curation service 1704 performs a process 17.04 to retrieve curation item details from entity tree 1708. Front end 1702 may then interact with entity tree 1706 via process 17.05 to retrieve any additional configuration information. In process 17.06, front end 1702 retrieves BPM XML files (e.g., for building a navigable graph, etc.) from BPMN engine 1706. In process 17.07, a query template is retrieved from entity tree 1708 by front end 1702. In process 17.08, front end 1702 retrieves process data from entity tree 1708. Process data includes, for example, treatment plan information, therapies and clinical trial information, consent forms, etc.

In process 17.09, patient data is retrieved from OIS services 1710 by front end 1702. Patient data may be in FHIR, COTA CNA, mCode, etc., formats. Front end 1702 then compiles all retrieved data to generate GUI elements and views as discussed above.

In process 17.10, front end 1702 messages BPM engine 1706 to start navigation and matching automations. In particular, curation item identifiers retrieved in in processes 17.03-17.04 and process and patient data retrieved in processes 17.09-17.10 are provided to BPM engine 1706 in process 17.10.

In process 17.11, front end 1702 retrieves process progress from BPM engine 1706. Process progress includes execution histories for pathways, either through BPM engine 1706 or provided as manual inputs via front end 1702. In cases where manual input is needed from the user to execute a decision node, the manual input is applied to the respective graph via process 17.12 from front end 1702 to BPM engine 1706. At process 17.13, front end 1702 sends an update to entity tree 1708. As a result, entity tree 1708 then reflects updated curation item status and can provide a correct and up to date data for curation items and processes (e.g., operations 17.05, 17.07, 17.08, etc.) upon request.

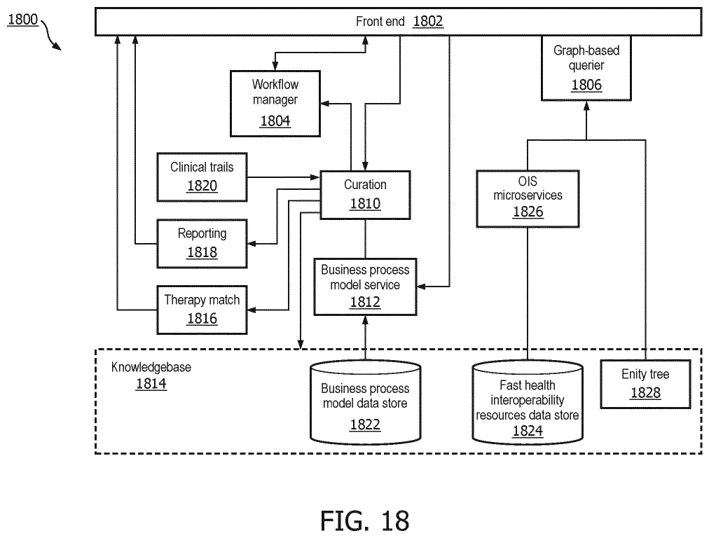

FIG. 18 is a systems diagram depicting a clinical support system 1800 which may include and/or perform the methods, systems, sequences, and features disclosed herein. Clinical support system 1800 includes a web front end 1802 which renders to a user pathways provider portal 104 and pathways authoring portal 106, and respective views therein. Front end 1802 renders GUI elements according to direction from a workflow manager 1804 and transmits back to backend components of clinical support system 1800 user inputs and the like. In some example, front 1802 includes authentication processes, either provided by clinical support system 1800 directly or via external API call. Based on user credentials (e.g., program manager role, practicing clinician role, administrator role, etc.), authenticated users may perform through front end 1802, for example and without imputing limitation, pathways authoring activities, treatment planning activities, pathway assignments, pathways updates, pathways execution and/or resumption, etc.

Workflow engine 1804 interacts with front end 1802, curation service 1810, therapy match service 1816, and reporting service 1818. In some examples, workflow engine 1804 interacts with additional services or may indirectly interact with a service via initiating downstream processes, etc. Workflow engine 1804 directs user interaction with clinical support system 1800 by sequentially retrieving patient input information from a fast health interoperability resources (FHIR) data store 1824, generating and navigating clinical pathways through either or both of user interaction (e.g., via user prompts, etc.) or automatic navigation via interfacing processes, selection of a recommendation from a terminal node within a clinical pathway, treatment plan generation, and consent form sign off and generation of reporting documents in an appropriate format (e.g., XML, PDF, JSON, etc.). Workflow manager 1804 integrates closely with front end 1802 components to present interactive applications to the user.

A knowledgebase 1814 supports various processes of clinical support system 1800, such as workflow manager 1804, curation service 1810, clinical trials service 1820, a business process model (BPM) data store 1822, an entity tree 1828, and, in some examples, FHIR data store 1824, in whole or in part. Entity tree 1828 provides a model, or template structure, for accessing and interacting with clinical pathways in the form of BPMN mappings (e.g., decision trees, graphs, etc.). In addition, a data model is stored in knowledge 1814 for supporting patient data standardization and transform where patient data is received (e.g., from FHIR data store 1824) in non-conformant formats. BPM store 1822 stores pathways in the form of BPMN networks.

BPM service 1812 matches representations (e.g., BPM networks in the form of decision trees, graphs, etc.) of pathways, or pathway components, with patient profiles. BPM service 1812 manages process workflow for authored pathway storage, pathway workflow initialization, why may then be handed off in whole or in part to workflow manager 1804, and process status notification.

Further, front end 1802 incorporates a graph-based querier 1806 (e.g., GraphQL, etc.) for interacting with FHIR data store 1824 via an OIC microservices bundle 1826 for retrieving and providing patient data. Graph-based querier 1806 generates queries based upon entity tree 1828 (e.g., conformant format, search parameters, etc.).

In some examples, with the exception of front end 1802 and graph-based querier 1806, the other components depicted in FIG. 18 are implemented in a cloud-based 0-footprint architecture. Data that is not needed for rendering a GUI is saved on a cloud, or hosted, server and front end 1802 is a web based application. A connection integrates local (e.g., clinic) infrastructure into the cloud-based architecture and data retrieved from local sources (e.g., PACS, RIS, EMR, etc.) is replicated to the cloud infrastructure to support the web based application as needed.

FIG. 19 depicts a relations and hierarchy structure 1900 between items curated by curation service 1810. Relations and hierarchy structure 1900 represents a model abstraction and it is understood that various data architectural paradigms may be used without departing from the spirit or scope of this disclosure.

Here, each pathway 1902 may be mapped to multiple treatment plans 1904 and multiple clinical trials 1906. Further, each treatment plan 1906 is mapped to a single respective regimen 1908. In effect, once an identifier for pathway 1902 has been established (e.g., a hash address, key value, etc.), mapped clinical trials 1906 and mapped treatment plans 1904, as well as respectively mapped regimen 1908, may be retrieved through relations and hierarchy structure 1900.

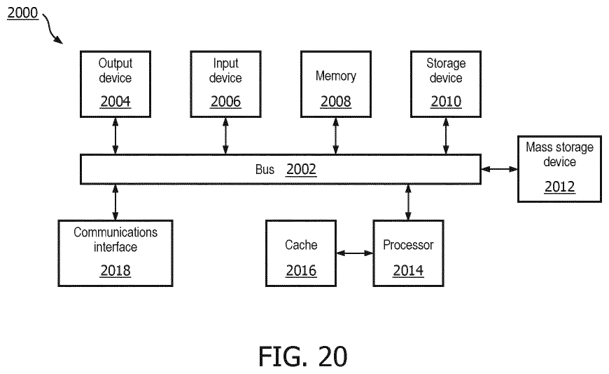

FIG. 20 is an example computing system 2000 that may implement various systems and methods discussed herein. The computer system 2000 includes one or more computing components in communication via a bus 2002. In one implementation, the computing system 2000 includes one or more processors 2014. The processor 2014 can include one or more internal levels of cache 2016 and a bus controller or bus interface unit to direct interaction with the bus 2002. The processor 2014 may specifically implement the various methods discussed herein. Main memory 2008 may include one or more memory cards and a control circuit (not depicted), or other forms of removable memory, and may store various software applications including computer executable instructions, that when run on the processor 2014, implement the methods and systems set out herein. Other forms of memory, such as a storage device 2010 and a mass storage device 2012, may also be included and accessible, by the processor (or processors) 2014 via the bus 2002. The storage device 2010 and mass storage device 2012 can each contain any or all of the methods and systems discussed herein.

The computer system 2000 can further include a communications interface 2018 by way of which the computer system 2000 can connect to networks and receive data useful in executing the methods and system set out herein as well as transmitting information to other devices. The computer system 2000 can also include an input device 2006 by which information is input. Input device 2006 can be a scanner, keyboard, and/or other input devices as will be apparent to a person of ordinary skill in the art. The computer system 2000 can also include an output device 2004 by which information can be output. Output device 2004 can be a monitor, printer, USB, and/or other output devices or ports as will be apparent to a person of ordinary skill in the art.

The system set forth in FIG. 20 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the methods can be rearranged while remaining within the disclosed subject matter. The accompanying method claims present elements of the various steps in a sample order and are not necessarily meant to be limited to the specific order or hierarchy presented.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A computer implemented method for generating a clinical pathway, the method comprising:
creating a new data object for representing the clinical pathway;
displaying a graphical tool to a user;
receiving, via the graphical tool, an identification of a first node to add to the clinical pathway;
updating the graphical tool to include a graphical representation of the first node;
receiving, via the graphical tool, an identification of a patient status in association with the first node;
updating the data object to include data representing the first node and the identification of the patient status; and
storing the data object in a database in association with an identification of a disease for future retrieval.

2. The method of claim 1, further comprising:
receiving, via the graphical tool, an identification of a second node to add to the clinical pathway;
receiving, via the graphical tool, a first characteristic in association with the second node, wherein the first characteristic defines criteria for determining applicability of the second node to a patient; and
updating the data object to include data representing the second node and the first characteristic.

3. The method of claim 2, further comprising:
displaying, via the graphical tool, a menu for selecting criteria including a plurality of selectable criteria options;
wherein the step of receiving the first characteristics comprises receiving a selected criteria option from the plurality of selectable criteria options selected by the user on the menu for selecting criteria.

4. The method of claim 3, wherein displaying the menu for selecting criteria comprises:
retrieving a plurality of ontological values from an ontology; and
presenting the plurality of ontological values as at least a portion of the plurality of selectable criteria options.

5. The method of claim 1, further comprising:
receiving, via the graphical tool, an identification of an additional node to add to the clinical pathway;
receiving, via the graphical tool, an identification of a treatment in association with the additional node; and
updating the data object to include data representing the additional node and the identification of a treatment.

6. The method of claim 5, wherein the identification of a treatment includes an identification of a clinical trial.

7. The method of claim 1, further comprising:
submitting the data object to a review process, wherein the review process comprises one or more reviewing authorities one of approving the data object for publication or suggesting revisions to the data object;
wherein the data object is not published until the one or more reviewing authorities have approved.

8. The method of claim 1, further comprising:
receiving a discipline identification; and
updating the data object to include the discipline identification.

9. The method of claim 8, wherein the discipline identification comprises one of medical oncology or radiation oncology.

10. The method of claim 1, wherein the graphical tool comprises a computer assisted design (CAD) interface provided through a web front end.

11. The method of claim 1, further comprising:
accessing a worklist of pathways, regimen, and treatment plans associated with a user;
wherein the data object is saved to the worklist and the user accesses the data object through the worklist to generate additional updates.

12. A computer-implemented method for treating a patient with a clinical pathway, the method comprising:
accessing patient information comprising one or more of diagnostic information, medical history, or current status information of a patient;
determining a clinical pathway for treating the patient based on the accessed patient information;
generating a graph based on the determined clinical pathway, the graph comprising an initial node comprising a disease identifier, one or more decision nodes comprising one or more of patient characteristics, treatment conditions or pathway timing elements, and one or more terminating nodes comprising treatment plan information or a reference to a second graph associated with a second clinical pathway;
navigating the graph to a terminating node based on the accessed patient information; and
providing one or more treatment plan recommendations based on the navigated to terminating node.

13. The method of claim 12, further comprising:
identifying a field within a decision node that cannot be determined based on the accessed patient information;
pausing navigation of the graph;
prompting a user for the identified field; and
resuming navigation of the graph in response to receiving the prompted for identified field.

14. The method of claim 13, wherein the identified field comprises a genomics test result for the patient.

15. The method of claim 12, further comprising:
receiving a treatment plan selection from among the generated treatment plan recommendations; and
generating one or more of one or more consent forms or a report based on the treatment plan selection.

16. The method of claim 15, further comprising:
receiving edits to the one or more of one or more consent forms or the report based on the treatment plan selection, the edits comprising one or more of modifications to a signature field, a signature date field, a provider field, or a signature time field.

17. The method of claim 12, further comprising:
receiving an off-pathway treatment plan specification, the off-pathway treatment plan specification including a rationale for treating the patient off-pathway.

18. The method of claim 12, wherein the one or more treatment plan recommendations comprises one or more of a Federal Drug Administration (FDA) approved therapy, an investigational therapy, or a clinical trial.
